(19) United States
(12) Patent Application Publication
(10) Pub. No.: US 2025/0115659 A1
(43) Pub. Date: Apr. 10, 2025

(54) NOVEL PROTEINS AND NUCLEIC ACID SEQUENCES AND USE THEREOF IN THE PROPHYLAXIS AND/OR TREATMENT OF CONGENITAL MUSCULAR DYSTROPHIES

(71) Applicant: UNIVERSITÄT BASEL, Basel (CH)

(72) Inventors: Markus A. RÜEGG, Riehen (CH); Judith REINHARD, Kriens (CH)

(21) Appl. No.: 18/842,690

(22) PCT Filed: Feb. 27, 2023
(86) PCT No.: PCT/EP2023/054822

§ 371 Date: Aug. 29, 2024

(30) Foreign Application Priority Data
Mar. 2, 2022 (EP) 22159671.1

(51) Int. Cl. C07K 14/78; A61K 38/00; A61K 48/00; A61P 21/00; C07K 14/47; C12N 15/86

(52) U.S. Cl. CPC C07K 14/78; A61K 48/005; A61P 21/00; C07K 14/4703; C12N 15/86; A61K 38/00; C07K 2319/02; C12N 2750/14143

(57) ABSTRACT

The present invention relates to a modified recombinant agrin protein or a fragment thereof and a recombinant chimeric laminin-nidogen protein or a fragment thereof, to nucleic acid sequences encoding these proteins, to vectors comprising these nucleic acid sequences, to compositions comprising these proteins and to uses of these proteins in the prophylaxis and/or treatment of congenital muscular dystrophies, in particular (laminin-α2) LAMA2-related muscular dystrophy.

### FIELD OF THE INVENTION

The present invention relates to a modified recombinant agrin protein or a fragment thereof and a recombinant chimeric laminin-nidogen protein or a fragment thereof, to nucleic acid sequences encoding these proteins, to vectors comprising these nucleic acid sequences, to compositions comprising these proteins and to uses of these proteins in the prophylaxis and/or treatment of congenital muscular dystrophies, in particular (laminin-α2) LAMA2-related muscular dystrophy.

### BACKGROUND OF THE INVENTION

The extracellular matrix (ECM) stabilizes cells and tissues by providing solid-phase cell adhesion substrates, ultimately linking the ECM to the cytoskeleton of cells. The consequence of ECM linkage loss is particularly evident in heritable muscular dystrophies. Mutations in distinct laminins lead to severe diseases, caused by the disruption of the ECM-to-receptor-to-cytoskeletal linkage. Mutation in the genes coding for these proteins cause muscular dystrophies supporting the concept that a disruption of transverse linkages from the ECM to cytoskeleton results in a failure of sarcolemmal stability and muscle function.

Laminin-211 (α2β1γ1 chain composition; abbreviated Lm-211) is expressed in adult muscle and peripheral nerve. Mutations within the LAMA2 gene (MIM 156 225), located on human chromosome 6q2 (Helbling-Leclerc A, et al. (1995)—Mutations in the laminin-α2 chain gene (LAMA2) cause merosin-deficient congenital muscular dystrophy. Nat Genet 11:216-218), encoding the α2 chain of Laminin-211, cause a particular subtype of congenital muscular dystrophies (CMDs), which are characterized as early onset and often very severe (C. Jimenez-Mallebrera, S. C. Brown, C. A. Sewry, F. Muntoni. Congenital muscular dystrophy: molecular and cellular aspects, Cell. Mol. Life Sci. 62 (7-8) (2005) 809-823). The so-called LAMA2 (congenital) muscular dystrophy (or LAMA2 MD, or LAMA2 CMD, previously also called MDC1A) is the largest subgroup of CMDs. For example, in a recent study of 249 CMD patients in the United Kingdom, 93 patients (37.4%) carried LAMA2 mutations (Sgframeli et al., Congenital muscular dystrophies in the UK population: Clinical and molecular spectrum of a large cohort diagnosed over a 12-year period. Neuromuscul Disord 27 (2017) 793-803). Mutations are spread throughout the LAMA2 gene with the majority being nonsense mutations that result in a premature protein truncation to cause a severe, non-ambulatory dystrophy (Yurchenco P D, McKee K K, Reinhard J R and Rüegg M A. Laminin-deficient muscular dystrophy: Molecular pathogenesis and structural repair strategies. Matrix Biology, 2018; https://doi.org/10.1016/j.matbio.2017.11.009). Patients with such mutations lack any laminin-α2 staining in muscle biopsies but express similar amounts of the β1 and the γ1 laminin chains as detected in healthy controls. Diagnosis requires genetic confirmation.

The clinical manifestation of LAMA2 MD congenital muscular dystrophy, as summarized by Mercuri E, Bönnemann C, Muntoni F (Mercuri E, Bönnemann C G, Muntoni F. Muscular dystrophies. Lancet 2019; 394:2025-38), is already evident in the first few days or weeks after birth. Affected children present with a generalized hypotonia (floppy infant syndrome) and strong increase in the levels of creatinine kinase in the blood. Further characteristics include frequent inability to acquire independent sitting, standing or walking and failure to thrive, probably because of weakness in muscles needed for swallowing. Typically, cognitive function is normal although there are characteristic changes in the white matter and occurrence of epilepsy in about 20% of the patients. Cardiac involvement is also rare. Patients who receive optimal care usually survive into early adulthood.

In recent findings the effect of a miniaturized and designed version of portions of the ECM molecule agrin in laminin-α2 deficient mice with transgenic overexpression was assessed. It was demonstrated that this “mini-agrin” (abbreviated as “mag”) functionally substitutes the missing linkage between the cell-surface receptor α-dystroglycan and the extracellular laminin network. Expression of this artificial “mini-agrin” significantly prolonged the life span of laminin a2-deficient mice, improved the motor performance and the muscle histology (J. Moll, P. Barzaghi, S. Lin, G. Bezakova, H. Lochmüller, E. Engvall, U. Müller, M. A. Ruegg. An agrin minigene rescues dystrophic symptoms in a mouse model for congenital muscular dystrophy. Nature. 2001 Sep. 20; 413 (6853): 302-7.). Although this strategy confers benefits, at least as demonstrated in an animal model of LAMA2 MD, muscular dystrophy still progresses in mag-expressing LAMA2-deficient mice and life expectancy remains markedly shorter than in wild-type controls. Other publications have shown beneficial effects by transgenic expression of mini-agrin (Reinhard J R, Lin S, McKee K K, Meinen S, Stephanie S C, Sury M, Hobbs S, Maier G, Yurchenco P D, Rüegg MA; 2017. Linker proteins restore basement membrane and correct LAMA2-related muscular dystrophy in mice. Sci. Transl. Med. 9, eaal4649). However, in the cited publication, the nucleic acid sequences expressed in the dyW/dyW mouse model for LAMA2 MD resulted in mini-agrin versions that appeared on Western blots as two bands with an apparent relative molecular mass of 80 and 110 kDa, respectively (FIG. 6). The higher molecular weight band corresponds to the full-length mini-agrin protein, whereas the lower molecular weight band is the result of proteolytic cleavage, indicating protein degradation of the full-length mini-agrin protein. Such protein degradation might severely limit the potential therapeutic use of mini-agrin.

US2003/0224981 teaches a method of treating LAMA2 MD in a mammal involving the administration of a protein comprising at least one binding domain for laminin and at least one binding domain for α-dystroglycan or a substance that upregulates endogenous agrin. The application of a substance that upregulates endogenous agrin may cause toxicity as it has been shown that particular splice variants of agrin, which are only expressed in motor neurons, can cause de-stabilization of the neuromuscular junction (Lin S, Maj M, Bezakova G, Magyar J P, Brenner H R, Ruegg M A (2008) Muscle-wide secretion of a miniaturized form of neural agrin rescues focal neuromuscular innervation in agrin mutant mice. Proc Natl Acad Sci USA 105:11406-11411).

Thus, there is still the need to find protein/nucleic acid sequences which are suitable to reach the therapeutic goal in humans and more in particular constructs that are stable and can be efficiently and safely transferred into human patients affected by LAMA2 MD.

It is an object of the present invention to provide biological molecules and/or compositions for use in the treatment of congenital muscular diseases, in particular (laminin-«2) LAMA2-related muscular dystrophy.

### SUMMARY OF THE INVENTION

The present invention provides a modified recombinant agrin protein or a fragment thereof, in particular a mini-agrin protein, and a recombinant chimeric laminin-nidogen protein or a fragment thereof, in particular an αLNNd protein, nucleic acid sequences encoding these proteins, vectors comprising these nucleic acid sequences, and compositions comprising these proteins, nucleic acid sequences and vectors.

The present invention also provides said proteins, nucleic acid sequences encoding said proteins, vectors comprising said nucleic acid sequences, and compositions comprising said proteins, nucleic acid sequences and vectors for use in the prophylaxis and/or treatment of congenital muscular dystrophies.

It has been surprisingly found by the inventors of the present application that a recombinant agrin protein or a fragment thereof comprising at least the laminin globular domain 3 (LG3 domain) and the amino acid sequence connecting the EGF-like domain 4 (EG4 domain) and the LG3 domain, wherein at least one amino acid residue of the amino acid sequence connecting the EG4 domain and the LG3 domain of the recombinant agrin protein or the fragment thereof is modified, is resistant to protein cleavage and degradation.

### BRIEF DESCRIPTION OF THE DRAWINGS

FIG. 1 shows a schematic representation of the modular structure of an agrin protein. Abbreviations used are, SS: signal sequence; NtA: N-terminal agrin domain; FS1: follistatin-like domain 1; FS: follistatin-like domain; LE: laminin epidermal growth factor (EGF) domain; SEA: sperm protein, enterokinase and agrin domain; S/T: serine/threonine-rich region; EG1: EGF-like domain 1; EG2: EGF-like domain 2; EG3: EGF-like domain 3; EG4: EGF-like domain 4; LG1: laminin globular domain 1; LG2: laminin globular domain 2; LG3: laminin globular domain 3. Black stretches indicate amino acid sequences connecting the elements designated with abbreviations. Elements used in the recombinant agrin protein or a fragment thereof according to the present invention are abbreviated and numbered accordingly.

FIG. 2 shows a schematic representation of the modular structure of a laminin protein. Abbreviations used are, SS: signal sequence; LN: laminin N-terminal domain. LE1: laminin EGF-like domain 1; LE2: laminin EGF-like domain 2; LE3: laminin EGF-like domain 3; LE4: laminin EGF-like domain 4; LE: laminin EGF-like domain; L4: laminin domain IV; LG: laminin globular domain. Black stretches indicate amino acid sequences connecting the elements designated with abbreviations. Elements used in the recombinant chimeric laminin-nidogen protein or a fragment thereof according to the present invention are abbreviated and numbered accordingly.

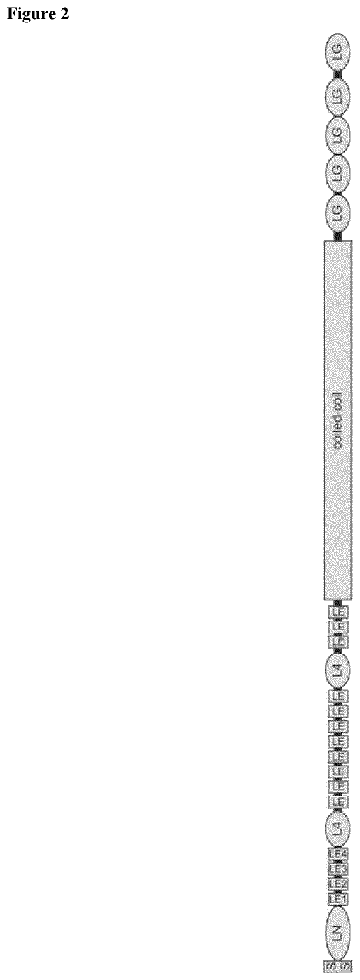

FIG. 3 shows a schematic representation of the modular structure of a nidogen protein. Abbreviations used are, SS: signal sequence; Ty: thyroglobulin type I repeat; G1: Globular nidogen domain 1; G2: Globular nidogen domain 2; G3: Globular nidogen domain 3; EG1: EGF-like domain 1; EG2: EGF-like domain 2; EG3: EGF-like domain 3; EG4: EGF-like domain 4; EG5: EGF-like domain 5. Black stretches indicate amino acid sequences connecting the elements designated with abbreviations. Elements used in the recombinant chimeric laminin-nidogen protein or a fragment thereof according to the present invention are abbreviated and numbered accordingly.

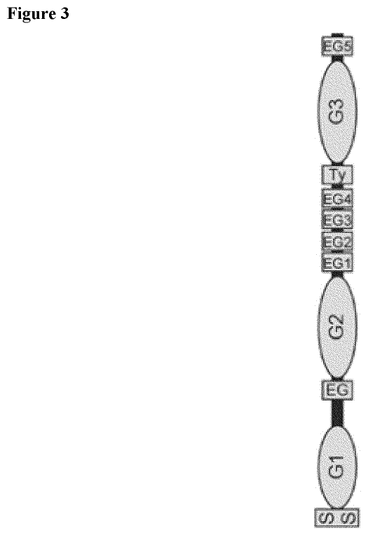

FIG. 4 shows a schematic representation of the modular structure of the fragment of the agrin protein according to the present invention. SS: signal sequence; NtA: N-terminal agrin domain; FS1: follistatin-like domain 1; EG1: EGF-like domain 1; EG2: EGF-like domain 2; EG3: EGF-like domain 3; EG4: EGF-like domain 4; LG1: laminin globular domain 1, LG2: laminin globular domain 2; LG3: laminin globular domain 3; wherein the stretch between the EG4 domain and the LG3 domain displayed corresponds to the modified amino acid sequence connecting the EG4 domain and the LG3 domain. Black stretches indicate amino acid sequences connecting the elements designated with abbreviations.

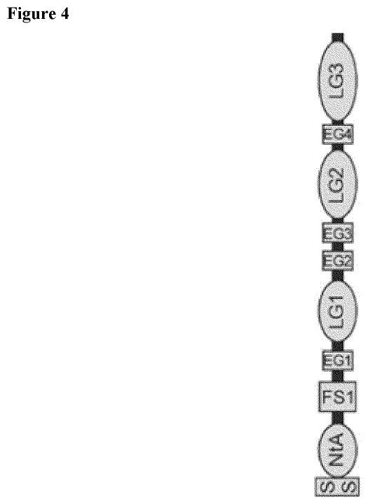

FIG. 5 shows a single letter amino acid sequence of human mini-agrin hmag974aa protein (SEQ ID NO:11). The box highlights the single amino acid substitution K187A from the carboxy terminus of the agrin protein or fragment thereof.

FIG. 6 shows a Western blot analysis to detect mini-agrin protein (mag) according to prior art (J. Moll, P. Barzaghi, S. Lin, G. Bezakova, H. Lochmüller, E. Engvall, U. Müller, M. A. Ruegg. An agrin minigene rescues dystrophic symptoms in a mouse model for congenital muscular dystrophy. Nature. 2001 Sep. 20; 413 (6853)) in lysates from triceps muscle from 8-week-old dyW/dyW mice transgenically expressing mag (compared to lysates from wild-type mice and dyW/dyW mice not expressing mag). GAPDH was used as loading control. Box: mag runs on SDS-PAGE as two bands indicating undesirable mini-agrin cleavage.

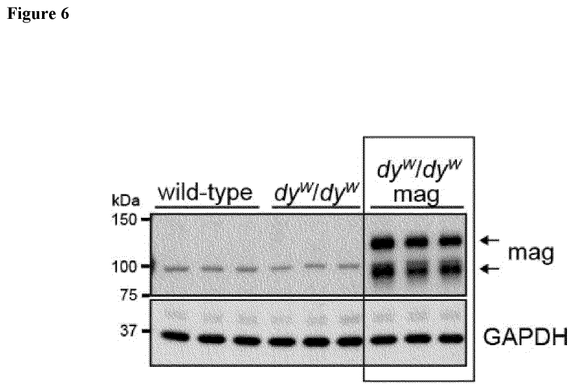

FIG. 7 shows a Western blot analysis to detect a mini-agrin protein (mag) according to prior art (J. Moll, P. Barzaghi, S. Lin, G. Bezakova, H. Lochmüller, E. Engvall, U. Müller, M. A. Ruegg. An agrin minigene rescues dystrophic symptoms in a mouse model for congenital muscular dystrophy. Nature. 2001 Sep. 20; 413 (6853)) in lysates from triceps muscle from 8-week-old mice transgenically expressing mag (left) or a hmag974aa protein according to the present invention with a single amino acid change at position 187 from the carboxy terminus, magK187A (right), representing the final hmag974aa construct. GAPDH was used as loading control. Lysates from wild-type (WT) mice not expressing mag are included for reference. For mag (left) only 51.7±4.2% is present as full-length protein (upper band); in contrast, magK187A (right) is not proteolytically cleaved.

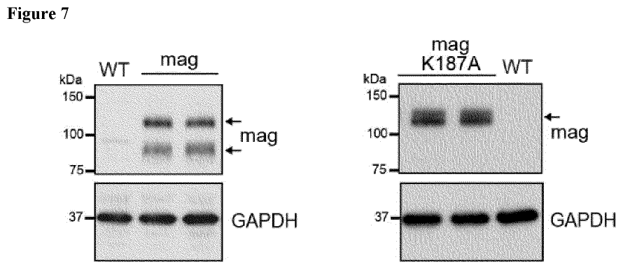

FIG. 8 shows the cloning map of the mag2925 bp construct with promoter sequence (Spc5.12), bovine growth hormone (BGH) polyadenylation signal (polyA), 5′ and 3′ inverted terminal (5′ITR, 3′ITR) repeat sequences for packaging into adeno-associated virus (AAV).

FIG. 9 shows a schematic representation of the modular structure of recombinant chimeric αLNNd according to the present invention. Abbreviations used are, SS: signal sequence; LN: laminin N-terminal domain; LE1: laminin EGF-like domain 1; LE2: laminin EGF-like domain 2; LE3: laminin EGF-like domain 3; LE4: laminin EGF-like domain 4; Ty: thyroglobulin type I repeat; EG1: EGF-like domain 1; EG2: EGF-like domain 2; EG3: EGF-like domain 3; EG4: EGF-like domain 4; EG5: EGF-like domain 5; G2: globular domain 2; G3: globular domain 3. Black stretches indicate amino acid sequences connecting the elements designated with abbreviations.

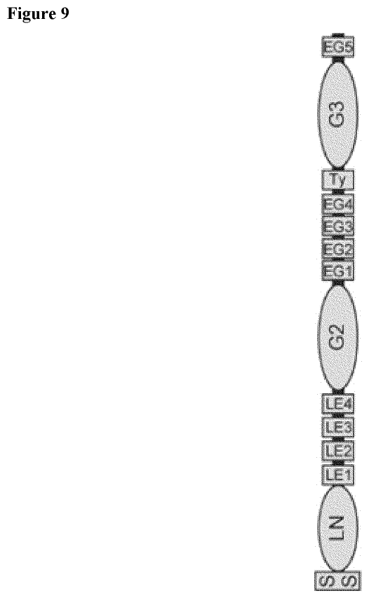

FIG. 10 shows a single letter amino acid sequence of hαLNNd1373aa protein (SEQ ID NO:39).

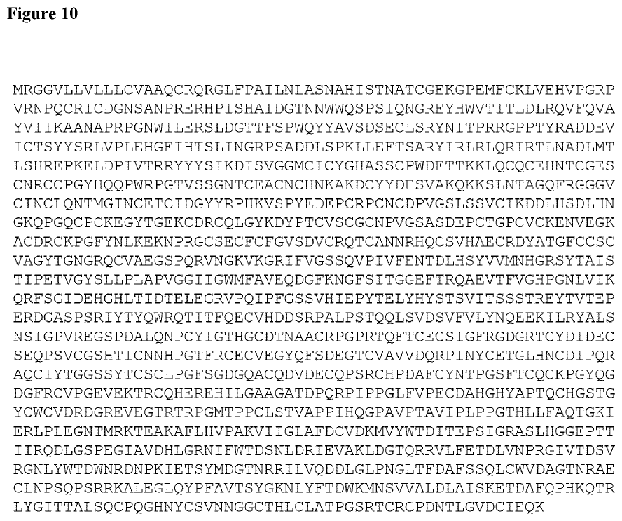

FIG. 11 shows the cloning map of the human «LNNd4122 bp construct with promoter sequence (Spc5.12), minimal polyA (pA), 5′ and 3′ inverted terminal repeat (5′ITR, 3′ITR) sequences for packaging into adeno-associated virus (AAV).

FIG. 12 shows the cloning map of the human αLNNd3009 bp construct with promoter sequence (CBh), BGH polyadenylation signal (polyA), 5′ and 3′ inverted terminal repeat (5′ITR, 3′ITR) sequences for packaging into adeno-associated virus (AAV).

FIG. 13 shows Western blot analysis (A) and immunochemistry (B) of an experiment showing AAV-mediated expression of proteins encoding hmag974aa and hαLNNd1373aa according to the present invention in mice.

FIG. 14 shows a graph depicting the effect on body weight of dyW/dyW mice from postnatal day 1 (P1) until postnatal day 56 (P56). At P1 mice were treated with AAV-mediated delivery of hmag974aa and hαLNNd1373aa (dyW AAV-DL) according to the invention.

FIG. 15 shows a graph depicting the effect on muscle weight of AAV-mediated delivery of hmag974aa and hαLNNd1373aa according to the invention.

FIG. 16 shows an image of histology (A) and a quantification of muscle fiber sizes (B) depicting the effect on muscle histology of AAV-mediated delivery of hmag974aa and hαLNNd1373aa according to the invention.

FIG. 17 shows quantifications depicting the effect on muscle function of AAV-mediated delivery of hmag974aa and hαLNNd1373aa according to the invention.

FIG. 18 shows a Kaplan-Meier survival curves depicting the effect on survival of AAV-mediated delivery of hmag974aa and hαLNNd1373aa according to the invention.

FIG. 19 shows quantifications of the effect on muscle weight of AAV-mediated delivery of hmag974aa and hαLNNd1373aa (both driven by the muscle-specific promoter Spc5.12) or hmag974aa (driven by Spc5.12) in combination with hαLNNd1002aa (driven by the ubiquitous promoter CBh).

FIG. 20 shows quantifications of the effect on gait performance of AAV-mediated hmag974aa (driven by Spc5.12) in combination with hαLNNd1002aa (driven by the ubiquitous promoter CBh).

FIG. 21 shows a single letter amino acid sequence of hαLNNd1002aa protein (SEQ ID NO:68).

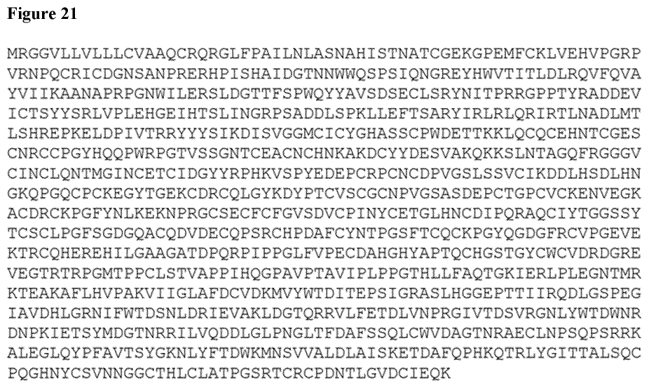

### DETAILED DESCRIPTION OF THE INVENTION

The present invention relates to a modified recombinant agrin protein or a fragment thereof and a recombinant chimeric laminin-nidogen protein or a fragment thereof, to nucleic acid sequences encoding these proteins, to vectors comprising these nucleic acid sequences, to compositions comprising these proteins and to uses of these proteins in the prophylaxis and/or treatment of congenital muscular dystrophies, in particular (laminin-α2) LAMA2-related muscular dystrophy.

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting. Features, integers, characteristics, compounds described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. Unless defined otherwise, all technical and scientific terms used herein have the same meaning as commonly understood by one of ordinary skill in the art to which this invention pertains.

The term “comprise” and variations thereof, such as, “comprises” and “comprising” is generally used in the sense of include, that is, as “including, but not limited to”, that is to say permitting the presence of one or more features or components.

The singular forms “a,” “an,” and “the” include plural referents unless the content clearly dictates otherwise.

The term “about” refers to a range of values±10% of a specified value. For example, the phrase “about 200” includes ±10% of 200, or from 180 to 220.

As used herein, the terms “polypeptide”, “peptide”, “protein”, “polypeptidic” and “peptidic” are used interchangeably to designate a series of amino acid residues connected to each other by peptide bonds between the alpha-amino and carboxy groups of adjacent residues. Preferably polypeptides have an amino acid sequence comprising at least 10 amino acids, more preferably at least 20 amino acids.

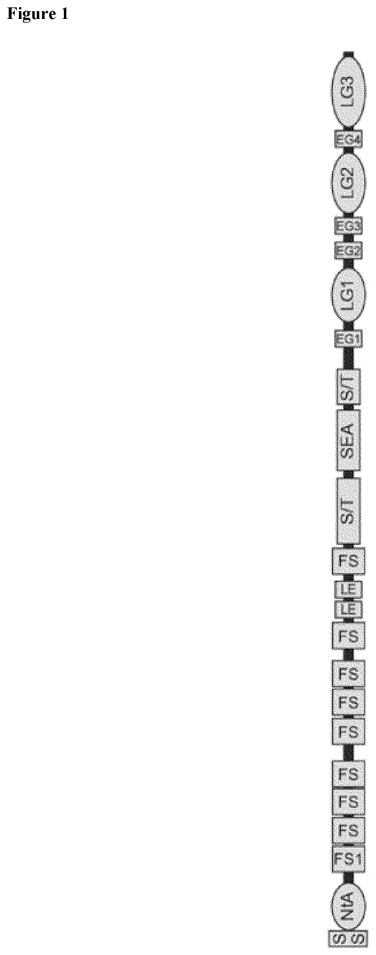

The term “agrin protein” as used herein refers to a large proteoglycan whose predominant role is in the development of the neuromuscular junction during embryogenesis which comprises the modular structure as shown in FIG. 1. Agrin is named based on its involvement in the aggregation of acetylcholine receptors during synaptogenesis. In humans, this protein is encoded by the AGRN gene. Preferably the agrin protein refers to the human agrin protein isoform 6 with the Uniprot (www.uniprot.org) number UniProtKB-O00468-6 (AGRIN_HUMAN). The full-length human agrin sequence is shown in SEQ ID NO:62. The term “wild-type agrin protein” refers to the naturally occurring agrin protein, preferably to the human agrin protein isoform 6 with the Uniprot (www.uniprot.org) number UniProtKB-000468-6 (AGRIN_HUMAN).

The term “a fragment of an agrin protein” or “fragment thereof” (as it relates to the agrin protein) as used herein refers to portion of the full-length agrin protein which usually has between 10 and 1500 amino acids, preferably between 50 and 1000 amino acids, more preferably between 100 and 1000 amino acids. A fragment of an agrin protein as defined herein does usually have the same functional properties as the agrin protein from which it is derived. The term “the agrin protein from which it is derived” in relation to a fragment refers to the full-length agrin protein from which the fragment is derived. The term “same functional properties as the agrin protein from which it is derived” refers to the molecular function (or one of the molecular functions) of the full-length protein from which the fragment is derived, which for example can be binding to laminin and to α-dystroglycan.

The term “at least one amino acid residue is modified” of a certain amino acid sequence as used herein refers to at least one amino acid substitution, insertion, and/or deletion in a polypeptide sequence. By “amino acid substitution” or “substitution” herein is meant the replacement of an amino acid at a particular position in a parent polypeptide sequence with another amino acid. For example, the substitution R94K refers to a variant polypeptide, in which the arginine at position 94 is replaced with a lysine. For example 94K indicates the substitution of position 94 with a lysine. For the purposes herein, multiple substitutions are typically separated by a slash or a comma. For example, “R94K/L78V” or “R94K, L78V” refers to a double variant comprising the substitutions R94K and L78V. By “amino acid insertion” or “insertion” as used herein is meant the addition of an amino acid at a particular position in a parent polypeptide sequence. For example, insert-94 designates an insertion at position 94. By “amino acid deletion” or “deletion” as used herein is meant the removal of an amino acid at a particular position in a parent polypeptide sequence. For example, R94-designates the deletion of arginine at position 94. Preferably, the term “at least one amino acid residue is modified”, refers to an amino acid substitution, more preferably to one amino acid substitution, even more preferably to one amino acid substitution at position 187 from the carboxy terminus of the recombinant agrin protein or the fragment thereof, wherein the numbering is according to isoform 6 of the agrin protein, in particular to amino acid substitution K187A from the carboxy terminus of the recombinant agrin protein or the fragment thereof. K187A from the carboxy terminus of the recombinant agrin protein or the fragment thereof means that the substitution of a lysine to an alanine occurs at the 187^{th} amino acid residue from the carboxy terminal end of the agrin protein or the fragment thereof.

The term “recombinant chimeric laminin-nidogen protein” as used herein refers to a fusion protein comprising at least the LN domain of laminin and the G3 domain of nidogen or comprising at least the LN domain of laminin and the G2 and G3 domain of nidogen and which can be produced by the joining of the respective parts of the two genes laminin (e.g. laminin-α1) and nidogen (e.g. nidogen-1). Laminins, essential components of the ECM, are heterotrimeric proteins that contain an α-chain, a β-chain, and a γ-chain. Preferably the laminin protein refers to the human laminin-α1 protein with the Uniprot (www.uniprot.org) number UniProtKB-P25391 (LAMA1_HUMAN). The full-length human laminin-α1 sequence is shown in SEQ ID NO:63. Nidogens, formerly known as entactins, are a family of glycoproteins located in the ECM. Preferably the nidogen protein refers to the human nidogen-1 protein with the Uniprot (www.uniprot.org) number UniProtKB-P14543 (NID1_HUMAN). The full-length human nidogen-1 sequence is shown in SEQ ID NO:64. The term “wild-type laminin protein” refers to the naturally occuring laminin protein, preferably to the human laminin-α1 protein with the Uniprot (www.uniprot.org) number UniProtKB-P25391 (LAMA1_HUMAN). The term “wild-type nidogen protein” refers to the naturally occuring nidogen protein, preferably to the human nidogen-1 protein with the Uniprot (www.uniprot.org) number UniProtKB-P14543 (NID1_HUMAN).

The term “a fragment of chimeric laminin-nidogen protein” as used herein refers to portion of the full-length chimeric laminin-nidogen protein which usually has between 10 and 1500 amino acids, preferably between 50 and 1000 amino acids, more preferably between 100 and 1000 amino acids. A fragment of a chimeric laminin-nidogen protein as defined herein does usually have the same functional properties as the laminin and the nidogen protein from which it is derived. The term “the chimeric laminin-nidogen protein from which it is derived” in relation to a fragment refers to the full-chimeric laminin-nidogen protein from which the fragment is derived. The term “same functional properties as the chimeric laminin-nidogen protein from which it is derived” refers to the molecular function (or one of the molecular functions) of the full-length protein from which the fragment is derived, which for example can be laminin polymerization and binding to the nidogen binding site of laminin, creating an artificial short arm for laminin polymerization.

The term “alpha LNNd protein” as used herein refers to laminin-nidogen protein or a fragment thereof comprising at least the LN domain of laminin and the G3 domain of nidogen or comprising at least the LN domain of laminin and the G2 and G3 domain of nidogen, and is most particular the alpha LNNd protein comprising the sequence as shown in SEQ ID NO:39 or the alpha LNNd protein comprising the sequence as shown in SEQ ID NO:68.

“Promoter” as used herein refers to a nucleic acid sequence that regulates expression of a transcriptional unit. A “promoter region” is a regulatory region capable of binding RNA polymerase in a cell and initiating transcription of a downstream (3′ direction) coding sequence. Within the promoter region will be found a transcription initiation site (conveniently defined by mapping with nuclease S1), as well as protein binding domains (consensus sequences) responsible for the binding of RNA polymerase such as the putative-35 region and the Pribnow box. The term “operably linked” when describing the relationship between two nucleotide e.g. DNA regions simply means that they are functionally related to each other and they are located on the same nucleic acid fragment. A promoter is operably linked to a structural gene if it controls the transcription of the gene and it is located on the same nucleic acid fragment as the gene. Useful promoter sequences are disclosed in the following documents. Synthetic promoter Spc5.12 (Li, X., Eastman, E. M., Schwartz, R. J., and Draghia-Akli, R. (1999). Synthetic muscle promoters: activities exceeding naturally occurring regulatory sequences. Nature biotechnology 17, 241-245). Muscle creatine kinase (MCK, CK8, CK6) (Himeda, C. L., Chen, X., and Hauschka, S. D. (2011). Design and testing of regulatory cassettes for optimal activity in skeletal and cardiac muscles. Methods Mol Biol 709, 3-19). Truncated MCK (tMCK) (Wang, B., Li, J., Fu, F. H., Chen, C., Zhu, X., Zhou, L., Jiang, X., and Xiao, X. (2008). Construction and analysis of compact muscle-specific promoters for AAV vectors. Gene therapy 15, 1489-1499). Myosin heavy chain (MHC) (Pacak, C. A., Sakai, Y., Thattaliyath, B. D., Mah, C. S., and Byrne, B. J. (2008). Tissue specific promoters improve specificity of AAV9 mediated transgene expression following intra-vascular gene delivery in neonatal mice. Genet Vaccines Ther 6, 13). Hybrid version of MHC and MCK (MHCK7) (Salva, M. Z., Himeda, C. L., Tai, P. W., Nishiuchi, E., Gregorevic, P., Allen, J. M., Finn, E. E., Nguyen, Q. G., Blankinship, M. J., Meuse, L., et al. (2007). Design of tissue-specific regulatory cassettes for high-level rAAV-mediated expression in skeletal and cardiac muscle. Molecular therapy: the journal of the American Society of Gene Therapy 15, 320-329). Desmin (Pacak, C. A., Sakai, Y., Thattaliyath, B. D., Mah, C. S., and Byrne, B. J. (2008). Tissue specific promoters improve specificity of AAV9 mediated transgene expression following intra-vascular gene delivery in neonatal mice. Genet Vaccines Ther 6, 13). Cytomegalovirus (CMV) (Boshart, M., Weber, F., Jahn, G., Dorsch-Hasler, K., Fleckenstein, B., and Schaffner, W. (1985). A very strong enhancer is located upstream of an immediate early gene of human cytomegalovirus. Cell 41, 521-530). Hybrid versions of cytomegalovirus (CMV) enhancer and chicken beta-actin (CAG, CB, CBA, CBh) (Gray, S. J., Foti, S. B., Schwartz, J. W., Bachaboina, L., Taylor-Blake, B., Coleman, J., Ehlers, M. D., Zylka, M. J., McCown, T. J., and Samulski, R. J. (2011). Optimizing promoters for recombinant adeno-associated virus-mediated gene expression in the peripheral and central nervous system using self-complementary vectors. Human gene therapy 22, 1143-1153). Elongation factor 1 alpha (EF1alpha, EF1alpha short, EFS) (Sanjana, N. E., Shalem, O., and Zhang, F. (2014). Improved vectors and genome-wide libraries for CRISPR screening. Nat Methods 11, 783-784). Myelin protein zero (Mpz, P0) (Kao S. C., Wu H., Xie J., Chang C. P., Ranish J. A., Graef I. A., Crabtree G. R. Calcineurin/NFAT Signaling Is Required for Neuregulin-Regulated Schwann Cell Differentiation. Science. 2009; 323 (5914): 651-4).

The term “muscle-specific promoter” refers to a tissue-specific promoter only active in muscle fibers and includes the Spc5.12 promoter, the muscle creatine kinase (MCK, CK8, CK6) promoter, the truncated MCK (tMCK) promoter, the myosin heavy chain (MHC) promoter, the hybrid version of MHC and MCK (MHCK7) promoter, the desmin promoter. Muscle specific promoters used in the present invention are preferably selected from the group consisting of the Spc5.12 promoter, the muscle creatine kinase (MCK, CK8, CK6) promoter and the hybrid version of MHC and MCK (MHCK7) promoter, and are preferably the Spc5.12 promoter. The term “ubiquitous promoter” refers to a promoter active, preferably strongly active, in a majority of cells, cell states and tissues and includes the CBh promoter, CBA promoter, CAG promoter, CMV promoter and EFS promoter. Ubiquitous promoters used in the present invention are preferably selected from the group consisting of the CBh promoter, CBA promoter and EFS promoter and are more preferably the CBh promoter. The term “Schwann cell-specific promoter” refers to a tissue-specific promoter active, preferably strongly active, in Schwann cells of the peripheral nervous system. A preferred Schwann cell-specific promoter used in the present invention is the Mpz promoter (Mpz, P0).

The term “congenital muscular dystrophies” as used herein refers to a group of genetic degenerative muscle disease (muscular dystrophies) that become apparent at birth or within the first months of life. The most frequent congenital muscular dystrophies in Europe is LAMA2-related muscular dystrophy (LAMA2 MD), also called muscular dystrophy congenital type 1A (MDC1A or merosin-deficient CMD). It is caused by mutations in the LAMA2 gene encoding the α2 subunit of laminin-211 (Lm-211). Most LAMA2 MD patients show complete absence of laminin-α2, are hypotonic (floppy) at birth, fail to ambulate, and die in the first decades of life due to respiratory complication.

In a first aspect the present invention provides a recombinant agrin protein or a fragment thereof, wherein the fragment thereof comprises at least the laminin globular domain 3 (LG3 domain) and the amino acid sequence connecting the EGF-like domain 4 (EG4 domain) and the LG3 domain of the recombinant agrin protein, and wherein at least one amino acid residue of the amino acid sequence connecting the EG4 domain and the LG3 domain of the recombinant agrin protein or the fragment thereof is modified. Usually, the recombinant agrin protein or a fragment thereof is of human origin i.e. is the human agrin protein or a fragment thereof. Preferably, the recombinant agrin protein or a fragment thereof is a recombinant agrin protein or a fragment thereof, wherein the fragment thereof comprises at least the EGF-like domain 4 (EG4 domain), the laminin globular domain 3 (LG3 domain) and the amino acid sequence connecting the EGF-like domain 4 (EG4 domain) and the LG3 domain of the recombinant agrin protein, and wherein at least one amino acid residue of the amino acid sequence connecting the EG4 domain and the LG3 domain of the recombinant agrin protein or the fragment thereof is modified.

More preferably the recombinant agrin protein or a fragment thereof is a recombinant agrin protein or a fragment thereof, wherein the fragment thereof comprises at least a N-terminal agrin domain (NtA domain), the laminin globular domain 2 (LG2 domain), the laminin globular domain 3 (LG3 domain) and the amino acid sequence connecting the EGF-like domain 4 (EG4 domain) and the LG3 domain of the recombinant agrin protein, and wherein at least one amino acid residue of the amino acid sequence connecting the EG4 domain and the LG3 domain of the recombinant agrin protein or the fragment thereof is modified.

Even more preferably the recombinant agrin protein or a fragment thereof is a recombinant agrin protein or a fragment thereof, wherein the fragment thereof comprises at least a N-terminal agrin domain (NtA domain), the EGF-like domain 4 (EG4 domain), the laminin globular domain 2 (LG2 domain), the laminin globular domain 3 (LG3 domain) and the amino acid sequence connecting the EGF-like domain 4 (EG4 domain) and the LG3 domain of the recombinant agrin protein, and wherein at least one amino acid residue of the amino acid sequence connecting the EG4 domain and the LG3 domain of the recombinant agrin protein or the fragment thereof is modified.

In one embodiment, the recombinant agrin protein or a fragment thereof is a recombinant wild-type agrin protein or a fragment thereof, wherein the fragment thereof comprises at least the laminin globular domain 3 (LG3 domain) and the amino acid sequence connecting the EGF-like domain 4 (EG4 domain) and the LG3 domain of the wild-type agrin protein, and wherein the at least one amino acid residue of the amino acid sequence connecting the EG4 domain and the LG3 domain of the recombinant wild-type agrin protein or the fragment thereof is modified as compared to the amino acid sequence connecting the EG4 domain and the LG3 domain of the wild-type agrin protein. Preferably, the recombinant agrin protein or a fragment thereof is a recombinant wild-type agrin protein or a fragment thereof, wherein the fragment thereof comprises at least the EGF-like domain 4 (EG4 domain), the laminin globular domain 3 (LG3 domain) and the amino acid sequence connecting the EGF-like domain 4 (EG4 domain) and the LG3 domain of the wild-type agrin protein, and wherein the at least one amino acid residue of the amino acid sequence connecting the EG4 domain and the LG3 domain of the recombinant wild-type agrin protein or the fragment thereof is modified as compared to the amino acid sequence connecting the EG4 domain and the LG3 domain of the wild-type agrin protein.

More preferably, the recombinant agrin protein or a fragment thereof is a recombinant wild-type agrin protein or a fragment thereof, wherein the fragment thereof comprises at least at least a N-terminal agrin domain (NtA domain), the laminin globular domain 2 (LG2 domain), the laminin globular domain 3 (LG3 domain) and the amino acid sequence connecting the EGF-like domain 4 (EG4 domain) and the LG3 domain of the wild-type agrin protein, and wherein the at least one amino acid residue of the amino acid sequence connecting the EG4 domain and the LG3 domain of the recombinant wild-type agrin protein or the fragment thereof is modified as compared to the amino acid sequence connecting the EG4 domain and the LG3 domain of the wild-type agrin protein.

Even more preferably, the recombinant agrin protein or a fragment thereof is a recombinant wild-type agrin protein or a fragment thereof, wherein the fragment thereof comprises at least at least a N-terminal agrin domain (NtA domain), the EGF-like domain 4 (EG4 domain), the laminin globular domain 2 (LG2 domain), the laminin globular domain 3 (LG3 domain) and the amino acid sequence connecting the EGF-like domain 4 (EG4 domain) and the LG3 domain of the wild-type agrin protein, and wherein the at least one amino acid residue of the amino acid sequence connecting the EG4 domain and the LG3 domain of the recombinant wild-type agrin protein or the fragment thereof is modified as compared to the amino acid sequence connecting the EG4 domain and the LG3 domain of the wild-type agrin protein.

In a further embodiment, the recombinant agrin protein is a recombinant human agrin protein or a fragment thereof, wherein the fragment thereof comprises at least the laminin globular domain 3 (LG3 domain) and the amino acid sequence connecting the EGF-like domain 4 (EG4 domain) and the LG3 domain of the recombinant human agrin protein, and wherein the at least one amino acid residue of the amino acid sequence connecting the EG4 domain and the LG3 domain of the recombinant human agrin protein or the fragment thereof is modified as compared to the amino acid sequence connecting the EG4 domain and the LG3 domain of the human agrin protein. Preferably, the recombinant agrin protein is a recombinant human agrin protein or a fragment thereof, wherein the fragment thereof comprises at least the EGF-like domain 4 (EG4 domain), the laminin globular domain 3 (LG3 domain) and the amino acid sequence connecting the EGF-like domain 4 (EG4 domain) and the LG3 domain of the recombinant human agrin protein, and wherein the at least one amino acid residue of the amino acid sequence connecting the EG4 domain and the LG3 domain of the recombinant human agrin protein or the fragment thereof is modified as compared to the amino acid sequence connecting the EG4 domain and the LG3 domain of the human agrin protein.

More preferably, the recombinant agrin protein is a recombinant human agrin protein or a fragment thereof, wherein the fragment thereof comprises at least a N-terminal agrin domain (NtA domain), the laminin globular domain 2 (LG2 domain), the laminin globular domain 3 (LG3 domain) and the amino acid sequence connecting the EGF-like domain 4 (EG4 domain) and the LG3 domain of the recombinant human agrin protein, and wherein the at least one amino acid residue of the amino acid sequence connecting the EG4 domain and the LG3 domain of the recombinant human agrin protein or the fragment thereof is modified as compared to the amino acid sequence connecting the EG4 domain and the LG3 domain of the human agrin protein.

Even more preferably, the recombinant agrin protein is a recombinant human agrin protein or a fragment thereof, wherein the fragment thereof comprises at least a N-terminal agrin domain (NtA domain), the laminin globular domain 2 (LG2 domain), the EGF-like domain 4 (EG4 domain), the laminin globular domain 3 (LG3 domain) and the amino acid sequence connecting the EGF-like domain 4 (EG4 domain) and the LG3 domain of the recombinant human agrin protein, and wherein the at least one amino acid residue of the amino acid sequence connecting the EG4 domain and the LG3 domain of the recombinant human agrin protein or the fragment thereof is modified as compared to the amino acid sequence connecting the EG4 domain and the LG3 domain of the human agrin protein.

In a preferred embodiment, the recombinant agrin protein or a fragment thereof is a recombinant agrin protein or a fragment thereof, wherein the LG2 domain does not contain one or more amino acid insertions at the y-site.

In a preferred embodiment, the recombinant agrin protein or a fragment thereof is a recombinant agrin protein or a fragment thereof, wherein the LG3 domain does not contain one or more amino acid insertions at the z-site.

In a more preferred embodiment, the recombinant agrin protein or a fragment thereof is a recombinant agrin protein or a fragment thereof, wherein the LG2 domain comprises the amino acid sequence LGESPVPHTV as shown in SEQ ID NO:70.

In a more preferred embodiment, the recombinant agrin protein or a fragment thereof is a recombinant agrin protein or a fragment thereof, wherein the LG3 domain comprises the amino acid sequence AVTESEKALQ as shown in SEQ ID NO:71.

In a preferred embodiment, the at least one amino acid residue of the amino acid sequence connecting the EG4 domain and the LG3 domain of the recombinant agrin protein or the fragment thereof which is modified is a lysine at position 187 from the carboxy terminus of the recombinant agrin protein or the fragment thereof. More preferably, the agrin protein or the fragment thereof is the isoform 6 of the agrin protein or a fragment thereof and the at least one amino acid residue of the amino acid sequence connecting the EG4 domain and the LG3 domain of the recombinant agrin protein or the fragment thereof which is modified is a lysine at position 187 from the carboxy terminus of the recombinant agrin protein or the fragment thereof,

In a further preferred embodiment, the lysine at position 187 from the carboxy terminus of the recombinant agrin protein or the fragment thereof is substituted with an amino acid with a nonpolar side chain selected from the group consisting of alanine, valine, leucine, isoleucine, phenylalanine, and methionine.

In a further preferred embodiment, the lysine at position 187 from the carboxy terminus of the recombinant agrin protein or the fragment thereof is substituted with alanine.

In a further embodiment, the amino acid sequence connecting the EG4 domain and the LG3 domain of the recombinant agrin protein or the fragment thereof comprises the amino acid sequence KGLVEASAGD as shown in SEQ ID NO:65.

In a further embodiment, the at least one modified amino acid residue of the amino acid sequence connecting the EG4 domain and the LG3 domain of the recombinant agrin protein or the fragment thereof is the A between E and S of the amino acid sequence KGLVEASAGD as shown in SEQ ID NO:65.

In a further embodiment, the fragment of the recombinant agrin protein further comprises the following polypeptides: a signal sequence (SS), a N-terminal agrin domain (NtA domain), a follistatin-like domain (FS domain), three EGF-like domains (EG domains) and two laminin globular domains (LG domains).

In a preferred embodiment, the fragment of the recombinant agrin protein is a mini-agrin comprising the following polypeptides: a signal sequence (SS), a N-terminal agrin domain (NtA domain), a follistatin-like domain (FS domain), EGF-like domain 1 (EG1 domain), laminin globular domain 1 (LG1 domain), EGF-like domain 2 (EG2 domain), EGF-like domain 3 (EG3 domain), laminin globular domain 2 (LG2 domain), EGF-like domain 4 (EG4 domain), the amino acid sequence connecting the EGF-like domain 4 (EG4 domain) and the LG3 domain and laminin globular domain 3 (LG3 domain), wherein at least one amino acid residue of the amino acid sequence connecting the EG4 domain and the LG3 domain of the fragment is modified.

In a further preferred embodiment, the fragment of the recombinant agrin protein is a mini-agrin comprising from the amino to the carboxy terminus: a signal sequence (SS), a N-terminal agrin domain (NtA domain), a follistatin-like domain (FS domain), EGF-like domain 1 (EG1 domain), laminin globular domain 1 (LG1 domain), EGF-like domain 2 (EG2 domain), EGF-like domain 3 (EG3 domain), laminin globular domain 2 (LG2 domain), EGF-like domain 4 (EG4 domain), the amino acid sequence connecting the EGF-like domain 4 (EG4 domain) and the LG3 domain and laminin globular domain 3 (LG3 domain), wherein at least one amino acid residue of the amino acid sequence connecting the EG4 domain and the LG3 domain of the fragment is modified.

In a more preferred embodiment, the recombinant agrin protein or a fragment thereof comprises:
the signal sequence (SS) comprising the sequence as shown in SEQ ID NO:1, and/or
the N-terminal agrin domain (NtA domain) comprising the sequence as shown in SEQ ID NO:2, and/or
the follistatin-like domain (FS domain) comprising the sequence as shown in SEQ ID NO:3, and/or
the EGF-like domain 1 (EG1 domain) comprising the sequence as shown in SEQ ID NO:4, the EGF-like domain 2 (EG2 domain) comprising the sequence as shown in SEQ ID NO:6, the EGF-like domain 3 (EG3 domain) comprising the sequence as shown in SEQ ID NO:7 and/or the EGF-like domain 4 (EG4 domain) comprising the sequence as shown in SEQ ID NO:9, and/or
the laminin globular 1 domain (LG1 domain) comprising the sequence as shown in SEQ ID NO:5, the laminin globular 2 domain (LG2 domain) comprising the sequence as shown in SEQ ID NO:8, the amino acid sequence connecting the EGF-like domain 4 (EG4 domain) and the LG3 domain as shown in SEQ ID NO:65 and/or the laminin globular 3 domain (LG3 domain) comprising the sequence as shown SEQ ID NO: 10.

In an even more preferred embodiment, the recombinant agrin protein or a fragment thereof comprises:
the signal sequence comprising the sequence as shown in SEQ ID NO:1,
the N-terminal agrin domain (NtA domain) comprising the sequence as shown in SEQ ID NO:2,
the follistatin-like domain (FS domain) comprising the sequence as shown in SEQ ID NO:3,
the EGF-like domain 1 (EG1 domain) comprising the sequence as shown in SEQ ID NO:4, the EGF-like domain 2 (EG2 domain) comprising the sequence as shown in SEQ ID NO:6, the EGF-like domain 3 (EG3 domain) comprising the sequence as shown in SEQ ID NO:7, the EGF-like domain 4 (EG4 domain) comprising the sequence as shown in SEQ ID NO:9,
the laminin globular 1 domain (LG1 domain) comprising the sequence as shown in SEQ ID NO:5, the laminin globular 2 domain (LG2 domain) comprising the sequence as shown in SEQ ID NO:8, the amino acid sequence connecting the EGF-like domain 4 (EG4 domain) and the LG3 domain as shown in SEQ ID NO:65 and the laminin globular 3 domain (LG3 domain) comprising the sequence as shown SEQ ID NO: 10.

In a particular embodiment the recombinant agrin protein or a fragment thereof comprises further amino acid sequences connecting the signal sequence, the sperm protein, enterokinase and agrin domain, the serine/threonine rich region and/or the domains as mentioned above, preferably comprises the amino acid sequences connecting the signal sequence, the sperm protein, enterokinase and agrin domain, the serine/threonine rich region and/or the domains as mentioned above of the wild-type agrin protein.

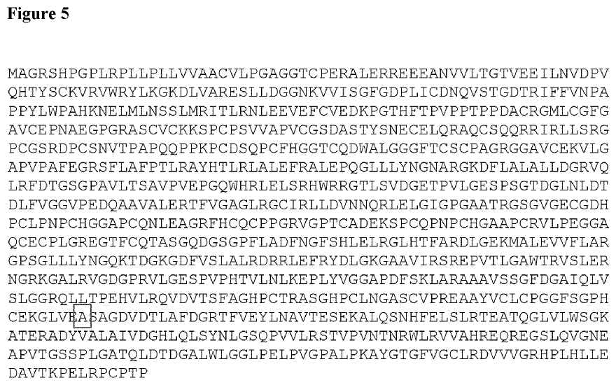

Most preferably the recombinant agrin protein or a fragment thereof comprises the sequence as shown in SEQ ID NO:11. This protein is a fragment of human agrin and will be referred to as hmag974aa herein. FIG. 5 shows the corresponding amino acid sequence of hmag974aa, the box indicating the single amino acid substitution K187A from the carboxy terminus of the agrin protein.

The present invention also relates to a nucleic acid sequence encoding the recombinant agrin protein or a fragment. In one embodiment the nucleic acid sequences comprises:
the signal sequence comprising the sequence as shown in SEQ ID NO:12, and/or
the N-terminal agrin domain comprising the sequence as shown in SEQ ID NO: 13, and/or
the follistatin-like domain comprising the sequence as shown in SEQ ID NO: 14, and/or
the EGF-like domain comprising the sequence as shown in SEQ ID NO:15, SEQ ID NO:17, SEQ ID NO: 18 and/or SEQ ID NO:20, and/or
the laminin globular domain comprising the sequence as shown in SEQ ID NO: 16, SEQ ID NO: 19 and/or SEQ ID NO:21.

In a preferred embodiment the nucleic acid sequence encoding the recombinant agrin protein or a fragment comprises:
the signal sequence comprising the sequence as shown in SEQ ID NO:12,
the N-terminal agrin domain comprising the sequence as shown in SEQ ID NO: 13,
the follistatin-like domain comprising the sequence as shown in SEQ ID NO: 14,
the EGF-like domain comprising the sequence as shown in SEQ ID NO:15, SEQ ID NO:17, SEQ ID NO:18 and/or SEQ ID NO:20,
the laminin globular domain comprising the sequence as shown in SEQ ID NO: 16, SEQ ID NO:19 and/or SEQ ID NO:21.

The nucleic acid sequence encoding the recombinant agrin protein or a fragment thereof according to the present invention most preferably comprises SEQ ID NO:22. This specific nucleic acid sequence will be referred to as hmag2925 bp in the following. hmag2925 bp encodes a specially designed mini-agrin protein with optimised codon usage. In a preferred embodiment, the nucleic acid sequence encoding the recombinant agrin protein or a fragment thereof, preferably the nucleic acid sequence encoding the recombinant agrin protein or a fragment thereof comprising SEQ ID NO:22, further comprises a muscle-specific promoter operably linked to the nucleic acid sequence, preferably the Spc5.12 promoter operably linked to the nucleic acid sequence.

In addition, to optimise the gene construct to allow efficient protein expression, the nucleic acid sequence encoding the recombinant agrin protein or a fragment thereof according to the present invention can be optimized for codon usage to ensure highest possible expression levels upon gene transfer in a human with LAMA2 MD. This can be achieved by a codon optimization process applying a freely available software tool (Puigbò P, Guzmá E, Romeu A, Garcia-Vallvé S. OPTIMIZER: a web server for optimizing the codon usage of DNA sequences. Nucleic Acids Res 2007). In addition, consensus Kozak sequences can be introduced into the gene construct. Codon usage optimisation is advantageous to (a) reduce the viral titres needed during the manufacturing process, as lower titres may reduce manufacturing costs and (b) to address safety concerns associated with high concentrations of viral vectors transferred into a human.

The present invention also relates to a vector comprising the above said nucleic acid sequences. The vector preferably further comprises, 5′ of the nucleic acid sequence, a promoter sequence of a muscle-specific promoter, an ubiquitous promoter or a Schwann cell-specific promoter, wherein the promoter sequence is more preferably a muscle-specific promoter selected from the group consisting of the synthetic promoter Spc5.12, the muscle creatine kinase (MCK, CK8, CK6) promoter, the truncated MCK (tMCK) promoter, the myosin heavy chain (MHC) promoter, the hybrid version of MHC and MCK (MHCK7) promoter, and the desmin promoter; an ubiquitous promoters selected from the group consisting of the cytomegalovirus (CMV) promoter, hybrid versions of cytomegalovirus (CMV) enhancer and chicken beta-actin promoter (CAG, CB, CBA, CBh), and the elongation factor 1 alpha (EF1alpha, EF1alpha short, EFS); or a Schwann cell-specific promoter, preferably the Myelin protein zero (Mpz, P0) promoter. The vector more preferably further comprises, 5′ of the nucleic acid sequence, a promoter sequence of a muscle-specific promoter, wherein the promoter sequence is more preferably selected from the group consisting of: the synthetic promoter Spc5.12, the muscle creatine kinase (MCK, CK8, CK6) promoter, the truncated MCK (tMCK) promoter, the myosin heavy chain (MHC) promoter, the hybrid version of MHC and MCK (MHCK7) promoter, the desmin promoter and is most preferably the Spc5.12 promoter. The selection of a suitable promoter depends on the tissue in which protein expression is most desirable, taking into account the known tissue-specificity of promoters. The above promoters are suitable for expression in muscle, cardiac and nervous tissue.

Preferred promoters useful for expression of the above described nucleic acid sequences are selected from the group consisting of Spc5.12 (SEQ ID NO:55), EFS (SEQ ID NO:56), CBh (SEQ ID NO:57), CK8 (SEQ ID NO:58), CK8e (SEQ ID NO: 59), CMV (SEQ ID NO:60), and MHCK7 (SEQ ID NO:61), Mpz (SEQ ID NO: 72), in particular the synthetic promoter Spc5.12 (SEQ ID NO:55).

In one embodiment, the vector further comprises a polyadenylation (polyA) sequence 3′ of the nucleic acid sequence and/or 5′ and 3′ inverted terminal repeat (5′ITR, 3′ITR) sequences respectively 5′ of the promoter and 3′ of the polyA sequence.

Preferred polyadenylation signal sequences for the expression of mag2925 bp are polyA from SV40 with length of 222 base pairs (bp), synthetic minimal polyA with length of 49 bp, or polyA from bovine-growth hormone (BGH) with length of 208 bp, the latter being the most preferred.

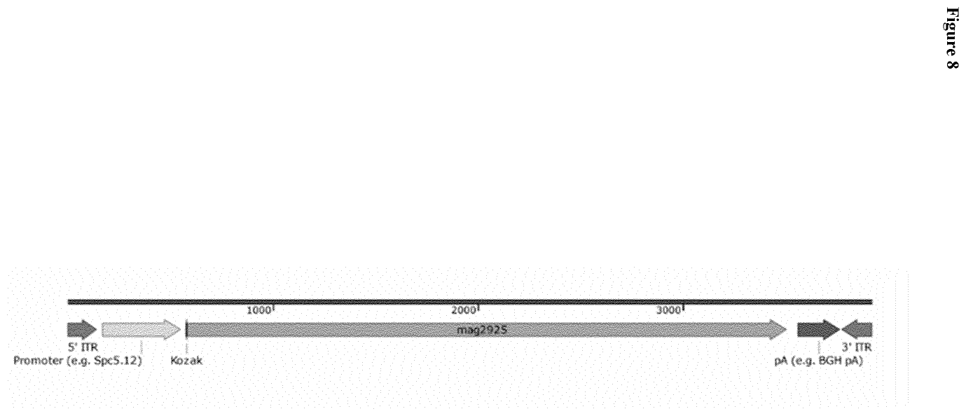

The cloning map of a preferred vector for mag2925 bp is shown in FIG. 8, with promoter sequence (Spc5.12), BGH polyadenylation signal (polyA), 5′ and 3′ inverted terminal repeat (5′ITR, 3′ITR) sequences for packaging into adeno-associated virus (AAV).

The vector is preferably an adeno-associated vector (AAV), more preferably AAV9, AAV8, AAVrh74, AAVMYO, AAVMYO2, AAVMYO3 or MyoAAV, even more preferably AAV9, AAV8, AAVrh74, AAVMYO or MyoAAV.

In a further aspect the present invention provides a recombinant chimeric laminin-nidogen protein or a fragment thereof, e.g. an alpha LNNd protein, comprising the sequence as shown in SEQ ID NO:39 or comprising the sequence as shown in SEQ ID NO: 68. This specific protein will be referred herein as hαLNNd1373aa or hαLNNd1002aa.

The present invention also relates to a nucleic acid sequence encoding the above said recombinant chimeric laminin-nidogen protein or a fragment thereof which preferably comprises the sequence as shown in SEQ ID NO:54 or the sequence as shown in SEQ ID NO:69. This specific nucleic acid sequence will be referred to as αLNNd4122 bp (SEQ ID NO:54) or αLNNd3009 bp (SEQ ID NO:69) in the following. In one embodiment, the nucleic acid sequence encoding the above said recombinant chimeric laminin-nidogen protein or a fragment thereof, preferably the nucleic acid sequence encoding the recombinant agrin protein or a fragment thereof comprising SEQ ID NO: 54 or SEQ ID NO:69, further comprises a muscle specific promoter or an ubiquitous promoter operably linked to the nucleic acid sequence, preferably the Spc5.12 promoter or the CBh promoter, more preferably the CBh promoter, operably linked to the nucleic acid sequence.

To optimise the gene construct to allow efficient protein expression, the nucleic acid sequence of the recombinant chimeric laminin-nidogen protein or a fragment thereof e.g. the αLNNd according to the present invention can be optimized for codon usage to ensure highest possible expression levels upon gene transfer in a human with LAMA2 MD. This can be done similarly to mini-agrin according to the present invention. In addition, consensus Kozak sequences can be introduced into the gene construct.

The present invention also relates to a vector comprising the above said nucleic acid sequence encoding a recombinant chimeric laminin-nidogen protein or a fragment thereof e.g. an alpha LNNd protein. The vector preferably further comprises, 5′ of the nucleic acid sequence, a promoter sequence of a muscle-specific promoter, an ubiquitous promoter or a Schwann cell-specific promoter, wherein the promoter sequence is more preferably a muscle-specific promoter selected from the group consisting of the synthetic promoter Spc5.12, the muscle creatine kinase (MCK, CK8, CK6) promoter, the truncated MCK (tMCK) promoter, the myosin heavy chain (MHC) promoter, the hybrid version of MHC and MCK (MHCK7) promoter, and the desmin promoter; an ubiquitous promoters selected from the group consisting of: the cytomegalovirus (CMV) promoter, hybrid versions of cytomegalovirus (CMV) enhancer and chicken beta-actin promoter (CAG, CB, CBA, CBh), and the elongation factor 1 alpha (EF1alpha, EF1alpha short, EFS); or a Schwann cell-specific promoter, preferably thze Myelin protein zero (Mpz, P0) promoter. The vector more preferably further comprises, 5′ of the nucleic acid sequence, a promoter sequence of a muscle-specific promoter or an ubiquitous promoter, wherein the promoter sequence is more preferably the Spc5.12 promoter or the CBh promoter, most preferably the CBh promoter. The selection of a suitable promoter depends on the tissue in which protein expression is most desirable, taking into account the known tissue-specificity of promoters. The above promoters are suitable for expression in muscle, cardiac and nervous tissue. References and sequences for the above said promoters have already been disclosed in the section relating to the agrin according to the present invention.

The vector further preferably comprises a polyadenylation (polyA) sequence 3′ of the nucleic acid sequence, and/or 5′ and 3′ inverted terminal repeat (5′ITR, 3′ITR) sequences respectively 5′ of the promoter and 3′ of the polyA sequence. Preferred polyadenylation signal sequences for the expression of αLNNd4122 bp are synthetic minimal polyA with length of 49 bp or synthetic polyA with length different from 49 bp as long as the chosen polyA tail enables the complete gene construct to be packed into a suitable viral gene transfer vector, the first being the most preferred. Preferred polyadenylation signal sequences for the expression hαLNNd3009 bp are polyA from SV40 with length of 222 base pairs (bp), synthetic minimal polyA with length of 49 bp, or polyA from bovine-growth hormone (BGH) with length of 208 bp, the latter being the most preferred.

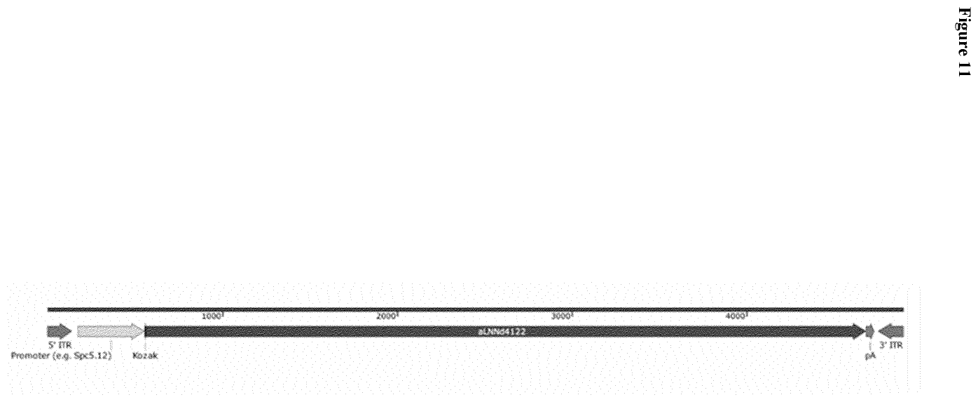

The cloning map of a preferred vector for αLNNd4122 bp is shown in FIG. 11, with promoter sequence (Spc5.12), minimal polyA (pA), 5′ and 3′ inverted terminal repeat (5′ITR, 3′ITR) sequences for packaging into adeno-associated virus (AAV).

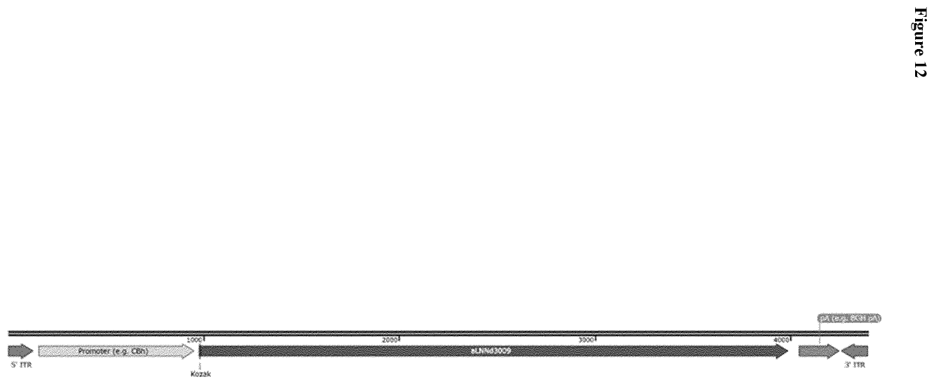

The cloning map of a preferred vector for αLNNd3009 bp is shown in FIG. 12 with promoter sequence (CBh), BGH polyadenylation signal (polyA), 5′ and 3′ inverted terminal repeat (5′ITR, 3′ITR) sequences for packaging into adeno-associated virus (AAV),

The vector is preferably an adeno-associated vector (AAV), more preferably AAV9, AAV8, AAVrh74, AAVMYO, AAVMYO2, AAVMYO3 or MyoAAV, even more preferably AAV9, AAV8, AAVrh74, AAVMYO, or MyoAAV.

In a further aspect the present invention provides a composition comprising the agrin protein or a fragment thereof as described above and a recombinant chimeric laminin-nidogen protein or a fragment thereof comprising at least the LN domain of laminin and the G3 domain of nidogen or comprising at least the LN domain of laminin and the G2 and G3 domain of nidogen.

In a preferred embodiment the recombinant chimeric laminin-nidogen protein or a fragment thereof, comprises the laminin (LN) binding domain of laminin (LN domain), and the globular domain 3 of nidogen (G3 domain) and further comprises a signal sequence, and/or a laminin EGF-like domain (LE domain), and/or an EGF-like domain (EG domain), and/or a thyroglobulin type I repeat (Ty).

In a further preferred embodiment the recombinant chimeric laminin-nidogen protein or a fragment thereof, comprises the laminin (LN) binding domain of laminin (LN domain), the globular domain 2 of nidogen (G2 domain), and the globular domain 3 of nidogen (G3 domain) and further comprises a signal sequence, and/or a laminin EGF-like domain (LE domain), and/or an EGF-like domain (EG domain), and/or a thyroglobulin type I repeat (Ty).

In a more preferred embodiment the recombinant chimeric laminin-nidogen protein or a fragment thereof, comprises from the amino to the carboxy terminus: a signal sequence, the laminin (LN) binding domain of laminin (LN domain), four laminin EGF-like domains (LE domain), two EGF-like domains (EG domains), a thyroglobulin type I repeat (Ty), the globular domain 3 of nidogen (G3 domain) and an EGF-like domain (EG domain).

In a further more preferred embodiment the recombinant chimeric laminin-nidogen protein or a fragment thereof, comprises from the amino to the carboxy terminus: a signal sequence, the laminin (LN) binding domain of laminin (LN domain), four laminin EGF-like domains (LE domain), the globular domain 2 of nidogen (G2 domain), four EGF-like domains (EG domains), a thyroglobulin type I repeat (Ty), the globular domain 3 of nidogen (G3 domain) and an EGF-like domain (EG domain).

In a particular embodiment the recombinant chimeric laminin-nidogen protein or a fragment thereof, comprises the laminin (LN) binding domain (LN domain) comprising the sequence as shown in SEQ ID NO:26, and the globular domain 3 of nidogen (G3 domain) comprising the sequence as shown in SEQ ID NO:37 and further comprises the signal sequence (SS) comprises SEQ ID NO:25, and/or the laminin EGF-like domain (LE domain) comprising the sequence as shown in SEQ ID NO:27 or SEQ ID NO: 28 or SEQ ID NO:29 or SEQ ID NO:30, and/or the EGF-like domain (EG domain) comprising the sequence as shown in SEQ ID NO:34 or SEQ ID NO:35, and/or the thyroglobulin type I repeat (Ty) comprising the sequence as shown in SEQ ID NO: 36, and/or the EGF-like domain (EG domain) comprising the sequence as shown in SEQ ID NO:38.

In a further particular embodiment the recombinant chimeric laminin-nidogen protein or a fragment thereof, comprises the laminin (LN) binding domain (LN domain) comprising the sequence as shown in SEQ ID NO:26, and the globular domain 3 of nidogen (G3 domain) comprising the sequence as shown in SEQ ID NO:37 and further comprises the signal sequence (SS) comprises SEQ ID NO:25, and/or the laminin EGF-like domain (LE domain) comprising the sequence as shown in SEQ ID NO:27 or SEQ ID NO: 28 or SEQ ID NO:29 or SEQ ID NO:30, and/or the globular domain 2 of nidogen (G2 domain) comprising the sequence as shown in SEQ ID NO:31, and/or the EGF-like domain (EG domain) comprising the sequence as shown in SEQ ID NO:32 or SEQ ID NO:33 or SEQ ID NO:34 or SEQ ID NO:35, and/or the thyroglobulin type I repeat (Ty) comprising the sequence as shown in SEQ ID NO:36, and/or the EGF-like domain (EG domain) comprising the sequence as shown in SEQ ID NO:38.

In a more particular embodiment the recombinant chimeric laminin-nidogen protein or a fragment thereof, comprises from the amino to the carboxy terminus:
the signal sequence comprising the sequence as shown in SEQ ID NO:25,
the laminin (LN) binding domain (LN domain) comprising the sequence as shown in SEQ ID NO:26,
the laminin EGF-like domain (LE domain) comprising the sequence as shown in SEQ ID NO:27, SEQ ID NO:28, SEQ ID NO:29, SEQ ID NO:30,
the EGF-like domain (EG domain) comprising the sequence as shown in SEQ ID NO: 34, SEQ ID NO:35,
the thyroglobulin type I repeat comprising the sequence as shown in SEQ ID NO: 36,
the globular domain 3 of nidogen (G3 domain) comprising the sequence as shown in SEQ ID NO:37, and
the EGF-like domain (EG domain) comprising SEQ ID NO:38.

In a further more particular embodiment the recombinant chimeric laminin-nidogen protein or a fragment thereof, comprises from the amino to the carboxy terminus:
the signal sequence comprising the sequence as shown in SEQ ID NO:25,
the laminin (LN) binding domain (LN domain) comprising the sequence as shown in SEQ ID NO:26,
the laminin EGF-like domain (LE domain) comprising the sequence as shown in SEQ ID NO:27, SEQ ID NO:28, SEQ ID NO:29, SEQ ID NO:30,
the globular domain 2 of nidogen (G2 domain) comprising the sequence as shown in SEQ ID NO:31,
the EGF-like domain (EG domain) comprising the sequence as shown in SEQ ID NO: 32, SEQ ID NO:33, SEQ ID NO:34, SEQ ID NO:35,
the thyroglobulin type I repeat comprising the sequence as shown in SEQ ID NO: 36,
the globular domain 3 of nidogen (G3 domain) comprising the sequence as shown in SEQ ID NO:37, and
the EGF-like domain (EG domain) comprising SEQ ID NO:38.

In a particular embodiment the recombinant chimeric laminin-nidogen protein or a fragment thereof comprises further amino acid sequences connecting the signal sequence, and/or the domains as mentioned above, preferably comprises the amino acid sequences connecting the signal sequence, and/or the domains as mentioned above of the wild-type laminin protein and of the wild-type nidogen protein, respectively.

In an even more particular embodiment the recombinant chimeric laminin-nidogen protein or a fragment thereof comprising at least the LN domain of laminin and the G2 and G3 domain of nidogen is an alpha LNNd protein, and is most particular the alpha LNNd protein comprising the sequence as shown in SEQ ID NO: 39 or the alpha LNNd protein comprising the sequence as shown in SEQ ID NO:68

The present invention also relates to a nucleic acid sequence encoding the recombinant chimeric laminin-nidogen protein or a fragment thereof comprising at least the LN domain of laminin and the G2 and G3 domain of nidogen is an alpha LNNd protein. In one embodiment the nucleic acid sequences comprises:
the signal sequence comprising the sequence as shown in SEQ ID NO:40,
the laminin (LN) binding domain (LN domain) comprising the sequence as shown in SEQ ID NO:41,
the laminin EGF-like domain (LE domain) comprising the sequence as shown in SEQ ID NO:43, SEQ ID NO:43, SEQ ID NO:44, SEQ ID NO:45,
the globular domain 2 of nidogen (G2 domain) comprising the sequence as shown in SEQ ID NO:46,
the EGF-like domain (EG domain) comprising the sequence as shown in SEQ ID NO: 47, SEQ ID NO:48, SEQ ID NO:49, SEQ ID NO:50,
the thyroglobulin type I repeat comprising the sequence as shown in SEQ ID NO: 51,
the globular domain 3 of nidogen (G3 domain) comprising the sequence as shown in SEQ ID NO:52, and
the EGF-like domain (EG domain) comprising SEQ ID NO:53.

In a preferred embodiment the composition comprises the agrin protein or a fragment thereof as described above and the recombinant chimeric laminin-nidogen protein or a fragment thereof comprising at least the LN domain of laminin and G3 domain of nidogen or comprising at least the LN domain of laminin and the G2 and G3 domain of nidogen as described above, preferably the agrin protein or a fragment thereof as described above and an alpha LNNd protein as described above, more preferably the agrin protein or a fragment thereof as described above and the alpha LNNd protein comprising the sequence as shown in SEQ ID NO: 39 or as shown in SEQ ID NO:68. Most preferred is a composition comprising the agrin protein or a fragment thereof comprising the sequence as shown in SEQ ID NO: 11 and the alpha LNNd protein comprising the sequence as shown in SEQ ID NO: 39 or as shown in SEQ ID NO:68.

Also provided by the present invention is a composition comprising the nucleic acid sequences encoding the agrin protein or a fragment thereof as described above and the nucleic acids encoding a recombinant chimeric laminin-nidogen protein or a fragment thereof comprising at least the LN domain of laminin and G3 domain of nidogen or comprising at least the LN domain of laminin and the G2 and G3 domain of nidogen as described above, or a the vector comprising the nucleic acid sequences encoding the agrin protein or a fragment thereof as described above and a vector comprising the nucleic acid sequences encoding a recombinant chimeric laminin-nidogen protein or a fragment thereof comprising at least the LN domain of laminin and G3 domain of nidogen or comprising at least the LN domain of laminin and the G2 and G3 domain of nidogen as described above, or a vector comprising the nucleic acid sequences encoding the agrin protein or a fragment thereof as described above and the nucleic acid sequences encoding a recombinant chimeric laminin-nidogen protein or a fragment thereof comprising at least the LN domain of laminin and G3 domain of nidogen or comprising at least the LN domain of laminin and the G2 and G3 domain of nidogen as described above, preferably wherein the vector is an AAV vector comprising these nucleic acid sequences. Most preferred is an AAV vector comprising the nucleic acid sequences as shown in SEQ ID NO: 22, as shown in SEQ ID NO: 54 or in SEQ ID NO: 69, in particular an AAV vector comprising the nucleic acid sequences as shown in SEQ ID NO: 22, as shown in SEQ ID NO: 54 or as shown in SEQ ID NO:69 and further comprising 5′ of the respective nucleic acid sequence, a promoter sequence as described above. The above disclosed proteins, nucleic acid sequences, vectors and/or compositions are effectively used in the prophylaxis and/or treatment of congenital muscular dystrophies.

Thus in a further aspect the present invention provides the agrin protein or a fragment thereof as described above and/or a recombinant chimeric laminin-nidogen protein or a fragment thereof comprising at least the LN domain of laminin and the G3 domain of nidogen or comprising at least the LN domain of laminin and the G2 and G3 domain of nidogen as described above or a composition comprising the agrin protein or a fragment thereof as described above and a recombinant chimeric laminin-nidogen protein or a fragment thereof comprising at least the LN domain of laminin and the G3 domain of nidogen or comprising at least the LN domain of laminin and the G2 and G3 domain of nidogen as described above, and the respective nucleic acid sequences and vectors as described above, for use in the prophylaxis and/or treatment of congenital muscular dystrophies. Likewise the present invention provides a method for prophylaxis and/or treatment of congenital muscular dystrophies in a subject, comprising administering to the subject the agrin protein or a fragment thereof as described above and/or a recombinant chimeric laminin-nidogen protein or a fragment thereof comprising at least the LN domain of laminin and the G3 domain of nidogen or comprising at least the LN domain of laminin and the G2 and G3 domain of nidogen as described above or the composition as described above, wherein the protein is administered in an amount that is sufficient to treat the subject.

Likewise the present invention provides to the use of the above described agrin protein or a fragment thereof and/or a recombinant chimeric laminin-nidogen protein or a fragment thereof comprising at least the LN domain of laminin and the G3 domain of nidogen or comprising at least the LN domain of laminin and the G2 and G3 domain of nidogen, or the composition as described above, for the manufacture of a medicament for prophylaxis and/or treatment of congenital muscular dystrophies in a subject. Likewise, the present invention provides the use of the above described agrin protein or a fragment thereof and/or a recombinant chimeric laminin-nidogen protein or a fragment thereof comprising at least the LN domain of laminin and the G3 domain of nidogen comprising at least the LN domain of laminin and the G2 and G3 domain of nidogen, or the composition as described above, for prophylaxis and/or treatment of congenital muscular dystrophies in a subject.

The congenital muscular dystrophy is preferably (laminin-α2) LAMA2-related muscular dystrophy. Preferably, the (laminin-α2) LAMA2-related muscular dystrophy patients express versions of Lm-211 in which the α2 chain is truncated at its N-terminal region that encodes the endogenous LN domain. In one embodiment, an AAV vector for expression of the above described agrin protein or a fragment thereof and an AAV vector for expression of and/or a recombinant chimeric laminin-nidogen protein or a fragment thereof comprising at least the LN domain of laminin and the G2 and G3 domain of nidogen or one vector for expression of both proteins are used in combination with immunosuppressants or omigapil (N-(dibenz(b,f)oxepin-10-ylmethyl)-N-methyl-N-prop-2-ynylamine).

The dosage regimens in a human subject are preferably from 1E13 to 3E14 vector genomes per kilogram (vg/kg) body weight of AAV vector for expression of the recombinant chimeric laminin-nidogen protein or a fragment thereof comprising at least the LN domain of laminin and the G3 domain of nidogen or comprising at least the LN domain of laminin and the G2 and G3 domain of nidogen and from 1E13 to 3E14 vector genomes per kilogram (vg/kg) body weight of AAV vector for expression of the recombinant agrin protein or a fragment thereof.

### EXAMPLES

### Example 1: Generation of Protein Constructs Encoding hmag974aa, hαLNNd1373aa and hαLNNd1002aa

Spc512 promoter and minimal polyA signal were generated by de novo DNA synthesis. hmag974aa (SEQ ID NO:11), hαLNNd1373aa (SEQ ID NO:39) and hαLNNd1002aa protein (SEQ ID NO:68) were codon optimized for human and generated by de novo DNA synthesis and inserted by standard restriction enzyme based cloning into AAV expression vectors including ITRs. The single amino acid substitution K187A from the carboxy terminus of the agrin protein fragment was generated by PCR-based site specific mutagenesis using oligo 5′-CCT GGT GGA GGC CAG CGC CGG CG-3 (SEQ ID NO:23) and oligo 5′-CCC TTC TCG CAG TGG GGG-3′ (SEQ ID NO:24).

### Example 2: Packaging of Protein Constructs into AAV Virus

AAV9 particles were produced by packaging cell line (HEK293T) by triple transfection using the recombinant AAV plasmids for protein expression as described in Example 1 together with a plasmid containing the essential rep and cap genes, and a third adenovirus-derived helper plasmid. The AAV particles were purified using IDX gradient ultracentrifugation to remove impurities and empty capsids. Viral titers were determined using SYBR Green based qPCR with primers targeting the ITR sequence (5′-GGAACCCCTAGTGATGGAGTT-3′ (SEQ ID NO:66) and 5′-CGGCCTCAGTGAGCGA-3′ (SEQ ID NO:67). Purity of AAV particles was assessed by 4-12% SDS-acrylamide gel electrophoresis and coomassie blue stain.

### Example 3: AAV-Mediated Expression of Proteins Encoding hmag974aa and hαLNNd1373aa in Mice

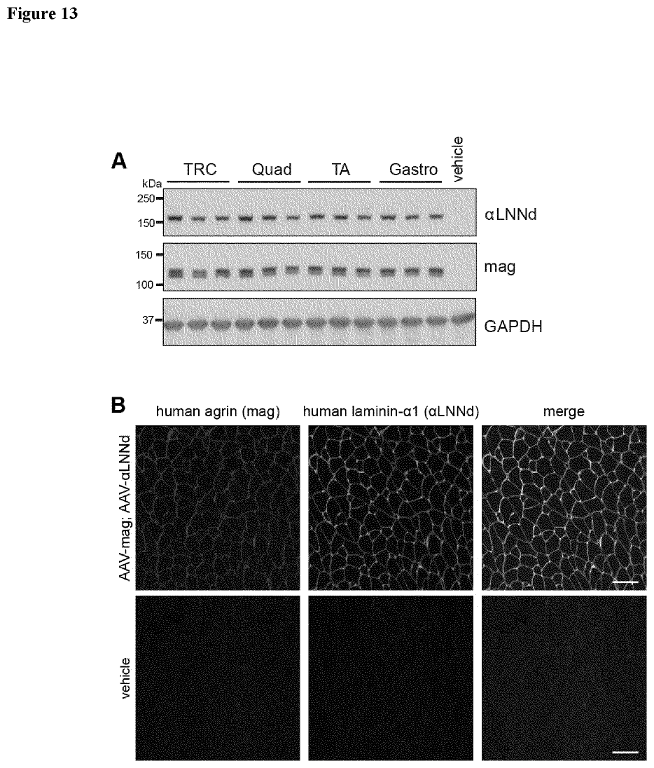

Purified AAV9 particles were diluted to a titer of 1E13 vector copies (vg)/ml in PBS. Newborn mice were co-injected at postnatal day 1 (P1) via the temporal vein with AAV9-hαLNNd1373aa (AAV-αLNNd) and AAV9-hmag974aa (AAV-mag) at a dose of 1.5E14 vg/kg (each vector). Eight weeks after injections muscles were collected and processed for Western blot analysis and immunohistochemistry. FIG. 13A shows that Western blot analysis confirmed presence of hmag974aa (mag) and hαLNNd1373aa in various muscles: triceps brachii (TRC), quadriceps (Quad), tibialis anterior (TA) and gastrocnemius (Gastro). GAPDH was used as loading control. FIG. 13B shows that immunohistochemistry of triceps brachii cross sections with antibodies against human agrin and human laminin-α1 confirmed presence of hmag974aa and hαLNNd1373aa protein in the extracellular matrix of muscle fibers. Scale bar, 100 μm.

### Example 4: Effect on Body Weight of Treatment with AAV-Mediated Delivery of hmag974aa and hαLNNd1373aa According to the Invention

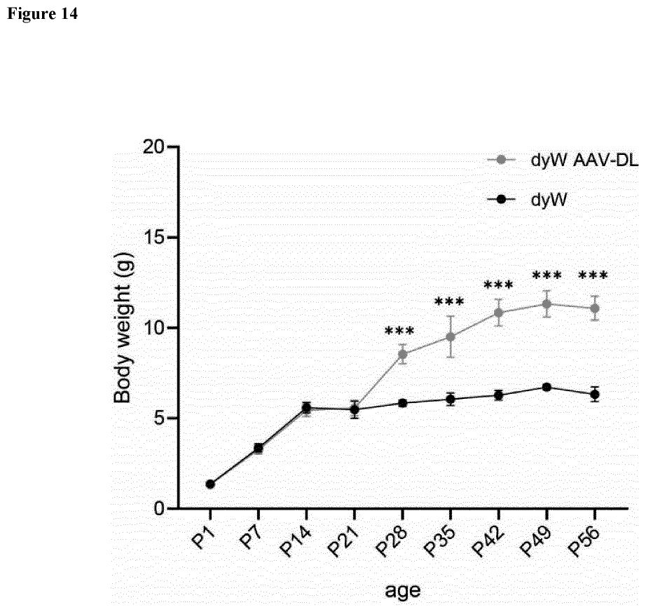

Treatment effect of AAV9-mediated delivery of hmag974aa and hαLNNd1373aa as described in Example 1-3 above was assessed in a mouse model for LAMA2-related muscular dystrophy (dyW mice). After genotyping at day of birth, mice were randomly assigned to treatment or vehicle group. One day after birth, mice were co-injected via the temporal vein with AAV9-hαLNNd1373aa and AAV9-hmag974aa at a dose of 1.5E14 vg/kg (each vector) (dyW AAV-DL) or vehicle only (dyW). Body weight was assessed once per week. Starting at 4 weeks of age, hmag974aa and hαLNNd1373aa treated dyW mice significantly increased body weight in contract to vehicle controls that fail to thrive. Results are shown in FIG. 14. Data are means±SEM. ***P<0.001. Student's t test. n=4-5 mice per group.

### Example 5: Effect on Muscle Weight of Treatment with AAV-Mediated Delivery of hmag974aa and hαLNNd1373aa According to the Invention

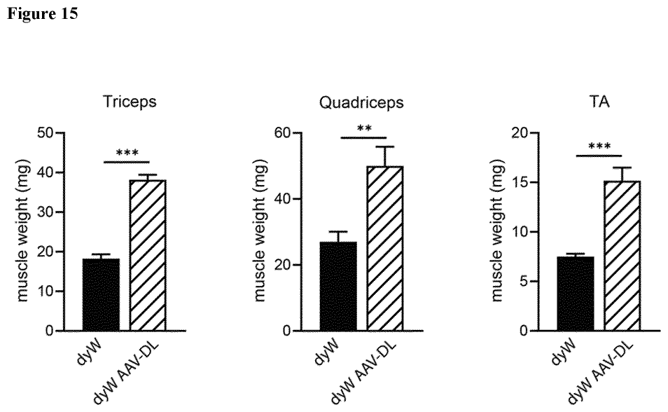

Treatment effect of AAV9-mediated delivery of hmag974aa and hαLNNd1373aa as described in Example 1-3 above was assessed in a mouse model for LAMA2-related muscular dystrophy (dyW mice). After genotyping at day of birth, mice were randomly assigned to treatment or vehicle group. One day after birth, mice were co-injected via the temporal vein with AAV9-hαLNNd1373aa and AAV9-hmag974aa at a dose of 1.5E14 vg/kg (each vector) (dyW AAV-DL) or vehicle only (dyW). Mice were euthanized at the age of 8 weeks and muscle tissue collected. AAV9-hαLNNd1373aa and AAV9-hmag974aa injection significantly increased muscle weights of all muscles analysed. Results are shown in FIG. 15. Data are means±SEM. ***P<0.001. **P<0.01. Student's t test. n=4-5 mice per group. TA: tibialis anterior muscle.

### Example 6: Effect on Muscle Histology of Treatment with AAV-Mediated Delivery of hmag974aa and hαLNNd1373aa According to the Invention

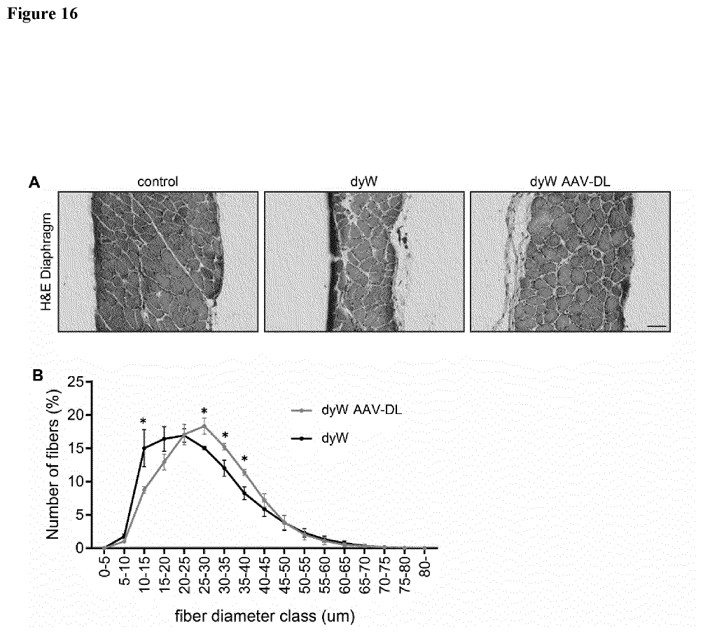

Treatment effect of AAV9-mediated delivery of hmag974aa and human hαLNNd1373aa as described in Example 1-3 above was assessed in a mouse model for LAMA2-related muscular dystrophy (dyW mice). After genotyping at day of birth, mice were randomly assigned to treatment or vehicle group. One day after birth, mice were co-injected via the temporal vein with AAV9-hαLNNd1373aa and AAV9-hmag974aa at a dose of 1.5E14 vg/kg (each vector) (dyW AAV-DL) or vehicle only (dyW). Mice were euthanized at the age of 8 weeks. FIG. 16A shows histology of diaphragm muscle, which was assessed by staining of cross sections with hematoxylin and eosin (H&E). Note that thickness of diaphragm and histological signs of muscular dystrophy was strongly improved by AAV9-hαLNNd1373aa and AAV9-hmag974aa injection. Scale bar, 50 μm. FIG. 16B shows a graph depicting distribution of the muscle fiber diameters of triceps muscles. Treatment with AAV9-hαLNNd1373aa and AAV9-hmag974aa significantly shifted fiber sizes towards larger fibers. Data are means±SEM. *P<0.05. n=3-4 mice per group.

### Example 7: Effect on Muscle Force of Treatment with AAV-Mediated Delivery of hmag974aa and hαLNNd1373aa According to the Invention

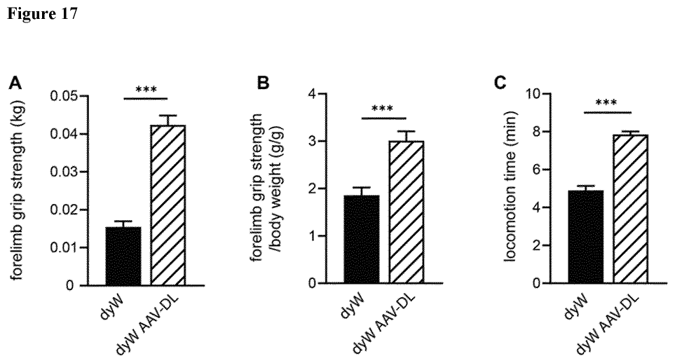

Treatment effect of AAV9-mediated delivery of hmag974aa and human hαLNNd1373aa as described in Example 1-3 above was assessed in a mouse model for LAMA2-related muscular dystrophy (dyW mice). After genotyping at day of birth mice were randomly assigned to treatment or vehicle group. One day after birth, mice were co-injected via the temporal vein with AAV9-hαLNNd1373aa and AAV9-hmag974aa at a dose of 1.5E14 vg/kg (each vector) (dyW AAV-DL) or vehicle only (dyW). At the age of 8 weeks muscle function was assessed. Total forelimb grip strength (A) and specific grip strength (B) were significantly increased by treatment with AAV9-hαLNNd1373aa and AAV9-hmag974aa. C) Voluntary locomotor activity was increased in treated dyW mice. Results are shown in FIG. 17. Data are means±SEM. ***P<0.001. Student's t test. n=5-8 mice per group.

### Example 8: Effect on Survival of Treatment with AAV-Mediated Delivery of hmag974aa and hαLNNd1373aa According to the Invention

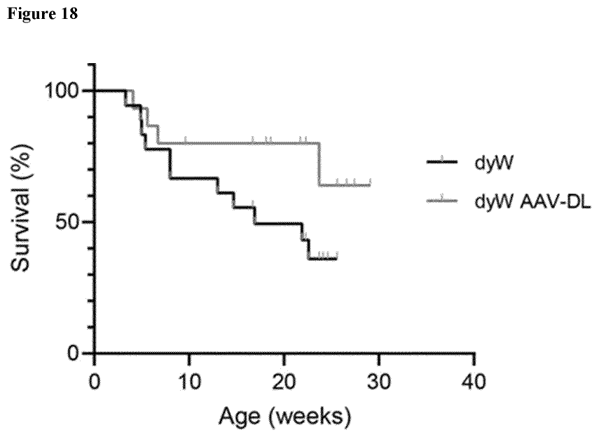

Treatment effect of AAV9-mediated delivery of hmag974aa and hαLNNd1373aa as described in Example 1-3 above was assessed in a mouse model for LAMA2-related muscular dystrophy (dyW mice). After genotyping at day of birth, mice were randomly assigned to treatment or vehicle group. One day after birth, mice were co-injected via the temporal vein with AAV9-hαLNNd1373aa and AAV9-hmag974aa at a dose of 1.5E14 vg/kg (each vector) (dyW AAV-DL) or vehicle only (dyW). Life span of mice is shown as a Kaplan-Meier cumulative survival curve. Marks indicate mice that were still alive. AAV9-hαLNNd1373aa and AAV9-hmag974aa treated animals had a higher probability of survival. Results are shown in FIG. 18.

### Example 9: Effect on Muscle Weight of Treatment with AAV9-Mediated Delivery of Spc5.12-hmag974aa and Spc5.12-hαLNNd1373aa or Spc5.12-hmag974aa and CBh-hαLNNd1002daa According to the Invention

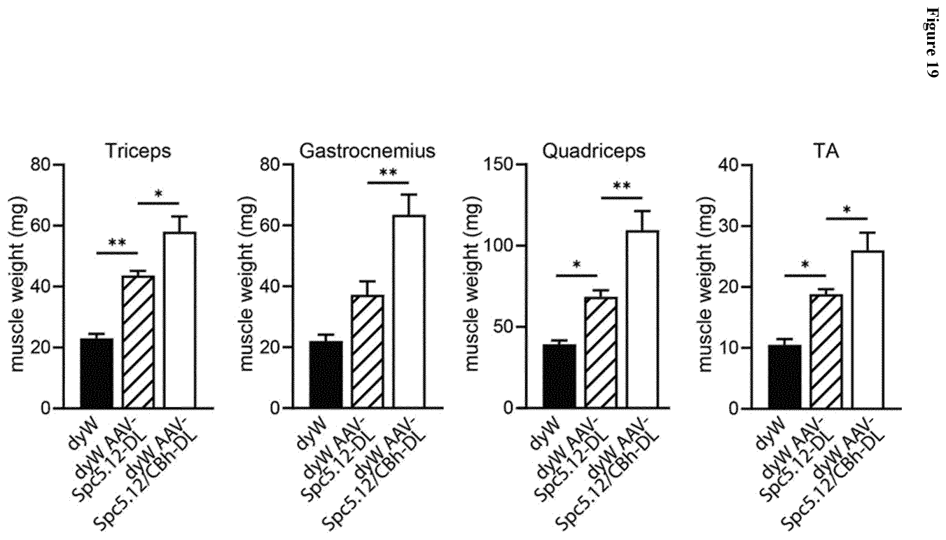

Treatment effect of AAV9-mediated delivery of hmag974aa and hαLNNd1373aa both driven by the muscle-specific promoter (Spc5.12) or by a hmag974aa (driven by the Spc5.12 promoter) and hαLNNd1002aa (driven by the ubiquitous promoter CBh) was assessed in a mouse model for LAMA2-related muscular dystrophy (dyW mice). After genotyping at day of birth, mice were randomly assigned to treatment or vehicle group. One day after birth, mice were co-injected via the temporal vein with AAV9-Spc5.12-hαLNNd1373aa and AAV9-Spc5.12-hmag974aa (dyW AAV-Spc5.12-DL) or AAV9-CBh-hαLNNd1002aa and AAV9-Spc5.12-hmag974aa (dyW AAV-Spc5.12/CBh-DL) at a dose of 1.5E14 vg/kg (each vector) or vehicle only (dyW). Mice were euthanized at the age of 8 weeks and muscle tissue collected. The specific combination of Spc5.12 promoter to drive hmag974aa and CBh for hαLNNd1002aa significantly increased muscle weight in comparison to muscle-specific expression. Results are shown in FIG. 19. Data are means±SEM. **P<0.01. *P<0.05. One-way ANOVA with Bonferroni post-hoc test. n=4-5 mice per group. TA: tibialis anterior muscle.

### Example 10: Effect on Gait of Treatment with AAV9-Mediated Delivery of Spc5.12-hmag974aa and CBh-hαLNNd1002daa According to the Invention

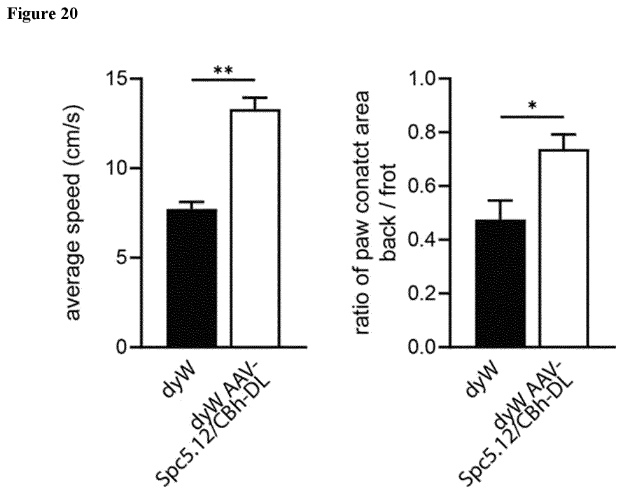

Treatment effect of AAV9-mediated delivery of hmag974aa (driven by Spc5.12) and hαLNNd1002 (driven by the ubiquitous promoter CBh) was assessed in a mouse model for LAMA2-related muscular dystrophy (dyW mice). After genotyping at day of birth, mice were randomly assigned to treatment or vehicle group. One day after birth, mice were co-injected via the temporal vein with AAV9-CBh-hαLNNd1002aa and AAV9-Spc5.12-hmag974aa (dyW AAV-Spc5.12/CBh-DL) at a dose of 1.5E14 vg/kg (each vector) or vehicle only (dyW). Gait performance was assessed at the age of 8 weeks. The specific combination of Spc5.12 promoter to drive hmag974aa and CBh for hαLNNd1002aa significantly increased gait performance of dyW mice. Results are shown in FIG. 20. Data are means±SEM. **P<0.01. *P<0.05. Student's t test. n=3-4 mice per group.

### Advantages

The recombinant agrin protein or a fragment thereof according to the present invention alone or in combination with the recombinant chimeric laminin/nidogen protein comprising at least the LN domain of laminin and the G3 domain of nidogen or comprising at least the LN domain of laminin and the G2 and G3 domain of nidogen according to the present invention, effectively allow polymerisation of laminin and stabilisation of muscle fibres by binding laminin to the cell membrane;

The recombinant agrin protein or a fragment thereof according to the present invention is considerably more stable than mini-agrin proteins according to the prior art.

The constructs for the recombinant agrin protein or a fragment thereof according to the present invention alone or in combination with the recombinant chimeric laminin/nidogen protein comprising at least the LN domain of laminin and the G3 domain of nidogen or comprising at least the LN domain of laminin and the G2 and G3 domain of nidogen according to the present invention are suitable to fit into AAV vectors and to be expressed efficiently.

The recombinant agrin protein or a fragment thereof according to the present invention alone or in combination with the recombinant chimeric laminin/nidogen protein comprising at least the LN domain of laminin and the G3 domain of nidogen or comprising at least the LN domain of laminin and the G2 and G3 domain of nidogen according to the present invention induce a low immunological response in vivo as they are made up of protein domains that are present in LAMA2 MD patients.

The recombinant agrin protein or a fragment thereof according to the present invention alone or in combination with the recombinant chimeric laminin/nidogen protein comprising at least the LN domain of laminin and the G3 domain of nidogen or comprising at least the LN domain of laminin and the G2 and G3 domain of nidogen according to the present invention are efficiently secreted and act outside of muscle fibres.

As shown in the in vivo experiments disclosed above, AAV-mediated delivery of the recombinant agrin protein or a fragment thereof according to the present invention alone or in combination with the recombinant chimeric laminin/nidogen protein comprising at least the LN domain of laminin and the G3 domain of nidogen or comprising at least the LN domain of laminin and the G2 and G3 domain of nidogen according to the invention significantly ameliorates the disease phenotype of the mouse model of LAMA2 MD by increasing body and muscle weight and improving muscle histology, fibre number, fibre size and lowering inflammatory response in muscle.

1. A recombinant agrin protein or a fragment thereof, wherein the fragment thereof comprises at least a N-terminal agrin domain (NtA domain), the laminin globular domain 2 (LG2 domain), the laminin globular domain 3 (LG3 domain) and the amino acid sequence connecting the EGF-like domain 4 (EG4 domain) and the LG3 domain of the recombinant agrin protein, and wherein at least one amino acid residue of the amino acid sequence connecting the EG4 domain and the LG3 domain of the recombinant agrin protein or the fragment thereof is modified, wherein the amino acid sequence connecting the EG4 domain and the LG3 domain of the recombinant agrin protein comprises the amino acid sequence KGLVEASAGD as shown in SEQ ID NO:65.

2. The recombinant agrin protein or a fragment thereof of claim 1, wherein the at least one modified amino acid residue of the amino acid sequence connecting the EG4 domain and the LG3 domain of the recombinant agrin protein or the fragment thereof is the A between E and S of the amino acid sequence KGLVEASAGD as shown in SEQ ID NO:65.

3. The recombinant agrin protein or a fragment thereof of any one of claims 1-2, wherein the fragment of the recombinant agrin protein further comprises the following polypeptides: a signal sequence (SS), a follistatin-like domain (FS domain), three EGF-like domains (EG domains) and one laminin globular domain (LG domain).

4. The recombinant agrin protein or a fragment thereof of any one of claims 1-2, wherein the fragment of the recombinant agrin protein comprises the following polypeptides: a signal sequence (SS), a N-terminal agrin domain (NtA domain), a follistatin-like domain (FS domain), EGF-like domain 1 (EG1 domain), laminin globular domain 1 (LG1 domain), EGF-like domain 2 (EG2 domain), EGF-like domain 3 (EG3 domain), laminin globular domain 2 (LG2 domain), EGF-like domain 4 (EG4 domain), the amino acid sequence connecting the EGF-like domain 4 (EG4 domain) and the LG3 domain and laminin globular domain 3 (LG3 domain), wherein at least one amino acid residue of the amino acid sequence connecting the EG4 domain and the LG3 domain of the fragment is modified, wherein the amino acid sequence connecting the EG4 domain and the LG3 domain of the recombinant agrin protein comprises the amino acid sequence KGLVEASAGD as shown in SEQ ID NO:65.

5. The agrin protein or a fragment thereof of any one of claims 1-4, comprising the sequence as shown in SEQ ID NO:11.

6. A nucleic acid sequence encoding an agrin protein or a fragment thereof of any one of claims 1-5.

7. The nucleic acid sequence according to claim 6, further comprising a muscle specific promoter operably linked to the nucleic acid sequence encoding an agrin protein or a fragment thereof.

8. A vector comprising the nucleic acid sequence according to claim 6 or claim 7.

9. A recombinant chimeric laminin-nidogen protein or a fragment thereof comprising the sequence as shown in SEQ ID NO: 39 or comprising the sequence as shown in SEQ ID NO: 68.

10. A nucleic acid sequence encoding a protein according to claim 9.

11. The nucleic acid sequence according to claim 10, further comprising a muscle specific promoter or a ubiquitous promoter operably linked to the nucleic acid sequence encoding a protein according to claim 9.

12. A vector comprising the nucleic acid sequence according to claim 10 or claim 11.

13. A composition comprising the agrin protein or a fragment thereof of any one of claims 1-5 and a recombinant chimeric laminin-nidogen protein or a fragment thereof comprising at least the LN domain of laminin and the G3 domain of nidogen or comprising at least the LN domain of laminin and the G2 and G3 domain of nidogen, preferably an alpha LNNd protein, more preferably the recombinant chimeric laminin-nidogen protein or a fragment thereof according to claim 9, or the nucleic acid sequence of claim 6 or 7 and the nucleic acid sequence of claim 10 or 11, or the vector of claim 8 and the vector of claim 12, or a vector comprising the nucleic acid sequence of claim 6 or 7 and the nucleic acid sequence of claim 10 or 11, preferably an AAV vector comprising the nucleic acid sequence of claim 6 or 7 and the nucleic acid sequence of claim 10 or 11.

14. The agrin protein or a fragment thereof of any one of claims 1-5 and/or the recombinant chimeric laminin-nidogen protein or a fragment thereof of claim 9, or the nucleic acid sequence of claim 6 or 7 and/or the nucleic acid sequence of claim 10 or 11, or the vector of claim 8 and/or the vector of claim 12, or the vector comprising the nucleic acid sequence of claim 6 or 7 and the nucleic acid sequence of claim 10 or 11, and/or the composition of claim 13 for use in the prophylaxis and/or treatment of congenital muscular dystrophies.

15. The proteins, nucleic acid sequences, vectors or the composition for use according to claim 14, wherein the congenital muscular dystrophy is (laminin-α2) LAMA2-related muscular dystrophy.

16. The proteins, nucleic acid sequences, vectors or the composition for use according to claim 14 or 15 wherein an AAV vector for expression of the recombinant laminin-nidogen protein and an AAV vector for expression of the agrin protein or a fragment thereof are used in combination with immunosuppressants or omigapil (N-(dibenz(b,f)oxepin-10-ylmethyl)-N-methyl-N-prop-2-ynylamine).


SEQUENCE LISTING (72 sequences)

SEQ ID NO: 1 - protein, 29 aa, Homo sapiens
MAGRSHPGPLRPLLPLLVVAACVLPGAGG

SEQ ID NO: 2 - protein, 128 aa, Homo sapiens
TCPERALERREEEANVVLTGTVEEILNVDPVQHTYSCKVRVWRYLKGKDLVARESLLDGGNKVVISGFGDPLICDNQVSTGDTRIFFVNPAPPYLWPAHKNELMLNSSLMRITLRNLEEVEFCVEDKP

SEQ ID NO: 3 - protein, 47 aa, Homo sapiens
VCKKSPCPSVVAPVCGSDASTYSNECELQRAQCSQQRRIRLLSRGPC

SEQ ID NO: 4 - protein, 35 aa, Homo sapiens
CDSQPCFHGGTCQDWALGGGFTCSCPAGRGGAVCE

SEQ ID NO: 5 - protein, 157 aa, Homo sapiens
APVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQ

SEQ ID NO: 6 - protein, 38 aa, Homo sapiens
CGDHPCLPNPCHGGAPCQNLEAGRFHCQCPPGRVGPTC

SEQ ID NO: 7 - protein, 38 aa, Homo sapiens
EKSPCQPNPCHGAAPCRVLPEGGAQCECPLGREGTFCQ

SEQ ID NO: 8 - protein, 184 aa, Homo sapiens
PFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPVPHTVLNLKEPLYVGGAPDFSKLARAAAVSSGFDGAIQLVSLGGRQLLTPEHVLRQVDVTSFAGHPC

SEQ ID NO: 9 - protein, 33 aa, Homo sapiens
ASGHPCLNGASCVPREAAYVCLCPGGFSGPHCE

SEQ ID NO: 10 - protein, 179 aa, Homo sapiens
VDTLAFDGRTFVEYLNAVTESEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPC

SEQ ID NO: 11 - protein, 974 aa, synthetic construct
MAGRSHPGPLRPLLPLLVVAACVLPGAGGTCPERALERREEEANVVLTGTVEEILNVDPVQHTYSCKVRVWRYLKGKDLVARESLLDGGNKVVISGFGDPLICDNQVSTGDTRIFFVNPAPPYLWPAHKNELMLNSSLMRITLRNLEEVEFCVEDKPGTHFTPVPPTPPDACRGMLCGFGAVCEPNAEGPGRASCVCKKSPCPSVVAPVCGSDASTYSNECELQRAQCSQQRRIRLLSRGPCGSRDPCSNVTPAPQQPPKPCDSQPCFHGGTCQDWALGGGFTCSCPAGRGGAVCEKVLGAPVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGECGDHPCLPNPCHGGAPCQNLEAGRFHCQCPPGRVGPTCADEKSPCQPNPCHGAAPCRVLPEGGAQCECPLGREGTFCQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPVPHTVLNLKEPLYVGGAPDFSKLARAAAVSSGFDGAIQLVSLGGRQLLTPEHVLRQVDVTSFAGHPCTRASGHPCLNGASCVPREAAYVCLCPGGFSGPHCEKGLVEASAGDVDTLAFDGRTFVEYLNAVTESEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 12 - DNA, 87 bp, synthetic construct
  source 1..87: mol_type = other DNA
atggccggcagaagccaccccggccccctgagacccctgctgcccctgctggtggtggccgcctgcgtgctgcccggcgccggcggc

SEQ ID NO: 13 - DNA, 384 bp, synthetic construct
  source 1..384: mol_type = other DNA
acctgccccgagagagccctggagagaagagaggaggaggccaacgtggtgctgaccggcaccgtggaggagatcctgaacgtggaccccgtgcagcacacctacagctgcaaggtgagagtgtggagatacctgaagggcaaggacctggtggccagagagagcctgctggacggcggcaacaaggtggtgatcagcggcttcggcgaccccctgatctgcgacaaccaggtgagcaccggcgacaccagaatcttcttcgtgaaccccgcccccccctacctgtggcccgcccacaagaacgagctgatgctgaacagcagcctgatgagaatcaccctgagaaacctggaggaggtggagttctgcgtggaggacaagccc

SEQ ID NO: 14 - DNA, 141 bp, synthetic construct
  source 1..141: mol_type = other DNA
gtgtgcaagaagagcccctgccccagcgtggtggcccccgtgtgcggcagcgacgccagcacctacagcaacgagtgcgagctgcagagagcccagtgcagccagcagagaagaatcagactgctgagcagaggcccctgc

SEQ ID NO: 15 - DNA, 105 bp, synthetic construct
  source 1..105: mol_type = other DNA
tgcgacagccagccctgcttccacggcggcacctgccaggactgggccctgggcggcggcttcacctgcagctgccccgccggcagaggcggcgccgtgtgcgag

SEQ ID NO: 16 - DNA, 471 bp, synthetic construct
  source 1..471: mol_type = other DNA
gcccccgtgcccgccttcgagggcagaagcttcctggccttccccaccctgagagcctaccacaccctgagactggccctggagttcagagccctggagccccagggcctgctgctgtacaacggcaacgccagaggcaaggacttcctggccctggccctgctggacggcagagtgcagctgagattcgacaccggcagcggccccgccgtgctgaccagcgccgtgcccgtggagcccggccagtggcacagactggagctgagcagacactggagaagaggcaccctgagcgtggacggcgagacccccgtgctgggcgagagccccagcggcaccgacggcctgaacctggacaccgacctgttcgtgggcggcgtgcccgaggaccaggccgccgtggccctggagagaaccttcgtgggcgccggcctgagaggctgcatcagactgctggacgtgaacaaccag

SEQ ID NO: 17 - DNA, 114 bp, synthetic construct
  source 1..114: mol_type = other DNA
tgcggcgaccacccctgcctgcccaacccctgccacggcggcgccccctgccagaacctggaggccggcagattccactgccagtgcccccccggcagagtgggccccacctgc

SEQ ID NO: 18 - DNA, 114 bp, synthetic construct
  source 1..114: mol_type = other DNA
gagaagagcccctgccagcccaacccctgccacggcgccgccccctgcagagtgctgcccgagggcggcgcccagtgcgagtgccccctgggcagagagggcaccttctgccag

SEQ ID NO: 19 - DNA, 552 bp, synthetic construct
  source 1..552: mol_type = other DNA
cccttcctggccgacttcaacggcttcagccacctggagctgagaggcctgcacaccttcgccagagacctgggcgagaagatggccctggaggtggtgttcctggccagaggccccagcggcctgctgctgtacaacggccagaagaccgacggcaagggcgacttcgtgagcctggccctgagagacagaagactggagttcagatacgacctgggcaagggcgccgccgtgatcagaagcagagagcccgtgaccctgggcgcctggaccagagtgagcctggagagaaacggcagaaagggcgccctgagagtgggcgacggccccagagtgctgggcgagagccccgtgccccacaccgtgctgaacctgaaggagcccctgtacgtgggcggcgcccccgacttcagcaagctggccagagccgccgccgtgagcagcggcttcgacggcgccatccagctggtgagcctgggcggcagacagctgctgacccccgagcacgtgctgagacaggtggacgtgaccagcttcgccggccacccctgc

SEQ ID NO: 20 - DNA, 99 bp, synthetic construct
  source 1..99: mol_type = other DNA
gccagcggccacccctgcctgaacggcgccagctgcgtgcccagagaggccgcctacgtgtgcctgtgccccggcggcttcagcggcccccactgcgag

SEQ ID NO: 21 - DNA, 537 bp, synthetic construct
  source 1..537: mol_type = other DNA
gtggacaccctggccttcgacggcagaaccttcgtggagtacctgaacgccgtgaccgagagcgagaaggccctgcagagcaaccacttcgagctgagcctgagaaccgaggccacccagggcctggtgctgtggagcggcaaggccaccgagagagccgactacgtggccctggccatcgtggacggccacctgcagctgagctacaacctgggcagccagcccgtggtgctgagaagcaccgtgcccgtgaacaccaacagatggctgagagtggtggcccacagagagcagagagagggcagcctgcaggtgggcaacgaggcccccgtgaccggcagcagccccctgggcgccacccagctggacaccgacggcgccctgtggctgggcggcctgcccgagctgcccgtgggccccgccctgcccaaggcctacggcaccggcttcgtgggctgcctgagagacgtggtggtgggcagacaccccctgcacctgctggaggacgccgtgaccaagcccgagctgagaccctgc

SEQ ID NO: 22 - DNA, 2925 bp, synthetic construct
  source 1..2925: mol_type = other DNA
atggccggcagaagccaccccggccccctgagacccctgctgcccctgctggtggtggccgcctgcgtgctgcccggcgccggcggcacctgccccgagagagccctggagagaagagaggaggaggccaacgtggtgctgaccggcaccgtggaggagatcctgaacgtggaccccgtgcagcacacctacagctgcaaggtgagagtgtggagatacctgaagggcaaggacctggtggccagagagagcctgctggacggcggcaacaaggtggtgatcagcggcttcggcgaccccctgatctgcgacaaccaggtgagcaccggcgacaccagaatcttcttcgtgaaccccgcccccccctacctgtggcccgcccacaagaacgagctgatgctgaacagcagcctgatgagaatcaccctgagaaacctggaggaggtggagttctgcgtggaggacaagcccggcacccacttcacccccgtgccccccaccccccccgacgcctgcagaggcatgctgtgcggcttcggcgccgtgtgcgagcccaacgccgagggccccggcagagccagctgcgtgtgcaagaagagcccctgccccagcgtggtggcccccgtgtgcggcagcgacgccagcacctacagcaacgagtgcgagctgcagagagcccagtgcagccagcagagaagaatcagactgctgagcagaggcccctgcggcagcagagacccctgcagcaacgtgacccccgccccccagcagccccccaagccctgcgacagccagccctgcttccacggcggcacctgccaggactgggccctgggcggcggcttcacctgcagctgccccgccggcagaggcggcgccgtgtgcgagaaggtgctgggcgcccccgtgcccgccttcgagggcagaagcttcctggccttccccaccctgagagcctaccacaccctgagactggccctggagttcagagccctggagccccagggcctgctgctgtacaacggcaacgccagaggcaaggacttcctggccctggccctgctggacggcagagtgcagctgagattcgacaccggcagcggccccgccgtgctgaccagcgccgtgcccgtggagcccggccagtggcacagactggagctgagcagacactggagaagaggcaccctgagcgtggacggcgagacccccgtgctgggcgagagccccagcggcaccgacggcctgaacctggacaccgacctgttcgtgggcggcgtgcccgaggaccaggccgccgtggccctggagagaaccttcgtgggcgccggcctgagaggctgcatcagactgctggacgtgaacaaccagagactggagctgggcatcggccccggcgccgccaccagaggcagcggcgtgggcgagtgcggcgaccacccctgcctgcccaacccctgccacggcggcgccccctgccagaacctggaggccggcagattccactgccagtgcccccccggcagagtgggccccacctgcgccgacgagaagagcccctgccagcccaacccctgccacggcgccgccccctgcagagtgctgcccgagggcggcgcccagtgcgagtgccccctgggcagagagggcaccttctgccagaccgccagcggccaggacggcagcggccccttcctggccgacttcaacggcttcagccacctggagctgagaggcctgcacaccttcgccagagacctgggcgagaagatggccctggaggtggtgttcctggccagaggccccagcggcctgctgctgtacaacggccagaagaccgacggcaagggcgacttcgtgagcctggccctgagagacagaagactggagttcagatacgacctgggcaagggcgccgccgtgatcagaagcagagagcccgtgaccctgggcgcctggaccagagtgagcctggagagaaacggcagaaagggcgccctgagagtgggcgacggccccagagtgctgggcgagagccccgtgccccacaccgtgctgaacctgaaggagcccctgtacgtgggcggcgcccccgacttcagcaagctggccagagccgccgccgtgagcagcggcttcgacggcgccatccagctggtgagcctgggcggcagacagctgctgacccccgagcacgtgctgagacaggtggacgtgaccagcttcgccggccacccctgcaccagagccagcggccacccctgcctgaacggcgccagctgcgtgcccagagaggccgcctacgtgtgcctgtgccccggcggcttcagcggcccccactgcgagaagggcctggtggaggccagcgccggcgacgtggacaccctggccttcgacggcagaaccttcgtggagtacctgaacgccgtgaccgagagcgagaaggccctgcagagcaaccacttcgagctgagcctgagaaccgaggccacccagggcctggtgctgtggagcggcaaggccaccgagagagccgactacgtggccctggccatcgtggacggccacctgcagctgagctacaacctgggcagccagcccgtggtgctgagaagcaccgtgcccgtgaacaccaacagatggctgagagtggtggcccacagagagcagagagagggcagcctgcaggtgggcaacgaggcccccgtgaccggcagcagccccctgggcgccacccagctggacaccgacggcgccctgtggctgggcggcctgcccgagctgcccgtgggccccgccctgcccaaggcctacggcaccggcttcgtgggctgcctgagagacgtggtggtgggcagacaccccctgcacctgctggaggacgccgtgaccaagcccgagctgagaccctgccccaccccctga

SEQ ID NO: 23 - DNA, 23 bp, synthetic construct
  source 1..23: mol_type = other DNA
cctggtggaggccagcgccggcg

SEQ ID NO: 24 - DNA, 18 bp, synthetic construct
  source 1..18: mol_type = other DNA
cccttctcgcagtggggg

SEQ ID NO: 25 - protein, 17 aa, Homo sapiens
MRGGVLLVLLLCVAAQC

SEQ ID NO: 26 - protein, 252 aa, Homo sapiens
RQRGLFPAILNLASNAHISTNATCGEKGPEMFCKLVEHVPGRPVRNPQCRICDGNSANPRERHPISHAIDGTNNWWQSPSIQNGREYHWVTITLDLRQVFQVAYVIIKAANAPRPGNWILERSLDGTTFSPWQYYAVSDSECLSRYNITPRRGPPTYRADDEVICTSYYSRLVPLEHGEIHTSLINGRPSADDLSPKLLEFTSARYIRLRLQRIRTLNADLMTLSHREPKELDPIVTRRYYYSIKDISVGGM

SEQ ID NO: 27 - protein, 57 aa, Homo sapiens
CICYGHASSCPWDETTKKLQCQCEHNTCGESCNRCCPGYHQQPWRPGTVSSGNTCEA

SEQ ID NO: 28 - protein, 70 aa, Homo sapiens
CNCHNKAKDCYYDESVAKQKKSLNTAGQFRGGGVCINCLQNTMGINCETCIDGYYRPHKVSPYEDEPCRP

SEQ ID NO: 29 - protein, 57 aa, Homo sapiens
CNCDPVGSLSSVCIKDDLHSDLHNGKQPGQCPCKEGYTGEKCDRCQLGYKDYPTCVS

SEQ ID NO: 30 - protein, 49 aa, Homo sapiens
CGCNPVGSASDEPCTGPCVCKENVEGKACDRCKPGFYNLKEKNPRGCSE

SEQ ID NO: 31 - protein, 238 aa, Homo sapiens
SPQRVNGKVKGRIFVGSSQVPIVFENTDLHSYVVMNHGRSYTAISTIPETVGYSLLPLAPVGGIIGWMFAVEQDGFKNGFSITGGEFTRQAEVTFVGHPGNLVIKQRFSGIDEHGHLTIDTELEGRVPQIPFGSSVHIEPYTELYHYSTSVITSSSTREYTVTEPERDGASPSRIYTYQWRQTITFQECVHDDSRPALPSTQQLSVDSVFVLYNQEEKILRYALSNSIGPVREGSPDA

SEQ ID NO: 32 - protein, 42 aa, Homo sapiens
LQNPCYIGTHGCDTNAACRPGPRTQFTCECSIGFRGDGRTCY

SEQ ID NO: 33 - protein, 42 aa, Homo sapiens
DIDECSEQPSVCGSHTICNNHPGTFRCECVEGYQFSDEGTCV

SEQ ID NO: 34 - protein, 44 aa, Homo sapiens
PINYCETGLHNCDIPQRAQCIYTGGSSYTCSCLPGFSGDGQACQ

SEQ ID NO: 35 - protein, 39 aa, Homo sapiens
DVDECQPSRCHPDAFCYNTPGSFTCQCKPGYQGDGFRCV

SEQ ID NO: 36 - protein, 74 aa, Homo sapiens
KTRCQHEREHILGAAGATDPQRPIPPGLFVPECDAHGHYAPTQCHGSTGYCWCVDRDGREVEGTRTRPGMTPPC

SEQ ID NO: 37 - protein, 287 aa, Homo sapiens
LSTVAPPIHQGPAVPTAVIPLPPGTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLTFDAFSSQLCWVDAGTNRAECLNPSQPSRRKALEGLQYPFAVTSYGKNLYFTDWKMNSVVALDLAISKETDAFQPHKQTRLYGITTALSQCP

SEQ ID NO: 38 - protein, 37 aa, Homo sapiens
GHNYCSVNNGGCTHLCLATPGSRTCRCPDNTLGVDCI

SEQ ID NO: 39 - protein, 1373 aa, synthetic construct
MRGGVLLVLLLCVAAQCRQRGLFPAILNLASNAHISTNATCGEKGPEMFCKLVEHVPGRPVRNPQCRICDGNSANPRERHPISHAIDGTNNWWQSPSIQNGREYHWVTITLDLRQVFQVAYVIIKAANAPRPGNWILERSLDGTTFSPWQYYAVSDSECLSRYNITPRRGPPTYRADDEVICTSYYSRLVPLEHGEIHTSLINGRPSADDLSPKLLEFTSARYIRLRLQRIRTLNADLMTLSHREPKELDPIVTRRYYYSIKDISVGGMCICYGHASSCPWDETTKKLQCQCEHNTCGESCNRCCPGYHQQPWRPGTVSSGNTCEACNCHNKAKDCYYDESVAKQKKSLNTAGQFRGGGVCINCLQNTMGINCETCIDGYYRPHKVSPYEDEPCRPCNCDPVGSLSSVCIKDDLHSDLHNGKQPGQCPCKEGYTGEKCDRCQLGYKDYPTCVSCGCNPVGSASDEPCTGPCVCKENVEGKACDRCKPGFYNLKEKNPRGCSECFCFGVSDVCRQTCANNRHQCSVHAECRDYATGFCCSCVAGYTGNGRQCVAEGSPQRVNGKVKGRIFVGSSQVPIVFENTDLHSYVVMNHGRSYTAISTIPETVGYSLLPLAPVGGIIGWMFAVEQDGFKNGFSITGGEFTRQAEVTFVGHPGNLVIKQRFSGIDEHGHLTIDTELEGRVPQIPFGSSVHIEPYTELYHYSTSVITSSSTREYTVTEPERDGASPSRIYTYQWRQTITFQECVHDDSRPALPSTQQLSVDSVFVLYNQEEKILRYALSNSIGPVREGSPDALQNPCYIGTHGCDTNAACRPGPRTQFTCECSIGFRGDGRTCYDIDECSEQPSVCGSHTICNNHPGTFRCECVEGYQFSDEGTCVAVVDQRPINYCETGLHNCDIPQRAQCIYTGGSSYTCSCLPGFSGDGQACQDVDECQPSRCHPDAFCYNTPGSFTCQCKPGYQGDGFRCVPGEVEKTRCQHEREHILGAAGATDPQRPIPPGLFVPECDAHGHYAPTQCHGSTGYCWCVDRDGREVEGTRTRPGMTPPCLSTVAPPIHQGPAVPTAVIPLPPGTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLTFDAFSSQLCWVDAGTNRAECLNPSQPSRRKALEGLQYPFAVTSYGKNLYFTDWKMNSVVALDLAISKETDAFQPHKQTRLYGITTALSQCPQGHNYCSVNNGGCTHLCLATPGSRTCRCPDNTLGVDCIEQK

SEQ ID NO: 40 - DNA, 51 bp, synthetic construct
  source 1..51: mol_type = other DNA
atgagaggcggcgtgctgctggtgctgctgctgtgcgtggccgcccagtgc

SEQ ID NO: 41 - DNA, 756 bp, synthetic construct
  source 1..756: mol_type = other DNA
agacagagaggcctgttccccgccatcctgaacctggccagcaacgcccacatcagcaccaacgccacctgcggcgagaagggccccgagatgttctgcaagctggtggagcacgtgcccggcagacccgtgagaaacccccagtgcagaatctgcgacggcaacagcgccaaccccagagagagacaccccatcagccacgccatcgacggcaccaacaactggtggcagagccccagcatccagaacggcagagagtaccactgggtgaccatcaccctggacctgagacaggtgttccaggtggcctacgtgatcatcaaggccgccaacgcccccagacccggcaactggatcctggagagaagcctggacggcaccaccttcagcccctggcagtactacgccgtgagcgacagcgagtgcctgagcagatacaacatcacccccagaagaggcccccccacctacagagccgacgacgaggtgatctgcaccagctactacagcagactggtgcccctggagcacggcgagatccacaccagcctgatcaacggcagacccagcgccgacgacctgagccccaagctgctggagttcaccagcgccagatacatcagactgagactgcagagaatcagaaccctgaacgccgacctgatgaccctgagccacagagagcccaaggagctggaccccatcgtgaccagaagatactactacagcatcaaggacatcagcgtgggcggcatg

SEQ ID NO: 42 - DNA, 171 bp, synthetic construct
  source 1..171: mol_type = other DNA
tgcatctgctacggccacgccagcagctgcccctgggacgagaccaccaagaagctgcagtgccagtgcgagcacaacacctgcggcgagagctgcaacagatgctgccccggctaccaccagcagccctggagacccggcaccgtgagcagcggcaacacctgcgaggcc

SEQ ID NO: 43 - DNA, 210 bp, synthetic construct
  source 1..210: mol_type = other DNA
tgcaactgccacaacaaggccaaggactgctactacgacgagagcgtggccaagcagaagaagagcctgaacaccgccggccagttcagaggcggcggcgtgtgcatcaactgcctgcagaacaccatgggcatcaactgcgagacctgcatcgacggctactacagaccccacaaggtgagcccctacgaggacgagccctgcagaccc

SEQ ID NO: 44 - DNA, 171 bp, synthetic construct
  source 1..171: mol_type = other DNA
tgcaactgcgaccccgtgggcagcctgagcagcgtgtgcatcaaggacgacctgcacagcgacctgcacaacggcaagcagcccggccagtgcccctgcaaggagggctacaccggcgagaagtgcgacagatgccagctgggctacaaggactaccccacctgcgtgagc

SEQ ID NO: 45 - DNA, 147 bp, synthetic construct
  source 1..147: mol_type = other DNA
tgcggctgcaaccccgtgggcagcgccagcgacgagccctgcaccggcccctgcgtgtgcaaggagaacgtggagggcaaggcctgcgacagatgcaagcccggcttctacaacctgaaggagaagaaccccagaggctgcagcgag

SEQ ID NO: 46 - DNA, 714 bp, synthetic construct
  source 1..714: mol_type = other DNA
agcccccagagagtgaacggcaaggtgaagggcagaatcttcgtgggcagcagccaggtgcccatcgtgttcgagaacaccgacctgcacagctacgtggtgatgaaccacggcagaagctacaccgccatcagcaccatccccgagaccgtgggctacagcctgctgcccctggcccccgtgggcggcatcatcggctggatgttcgccgtggagcaggacggcttcaagaacggcttcagcatcaccggcggcgagttcaccagacaggccgaggtgaccttcgtgggccaccccggcaacctggtgatcaagcagagattcagcggcatcgacgagcacggccacctgaccatcgacaccgagctggagggcagagtgccccagatccccttcggcagcagcgtgcacatcgagccctacaccgagctgtaccactacagcaccagcgtgatcaccagcagcagcaccagagagtacaccgtgaccgagcccgagagagacggcgccagccccagcagaatctacacctaccagtggagacagaccatcaccttccaggagtgcgtgcacgacgacagcagacccgccctgcccagcacccagcagctgagcgtggacagcgtgttcgtgctgtacaaccaggaggagaagatcctgagatacgccctgagcaacagcatcggccccgtgagagagggcagccccgacgcc

SEQ ID NO: 47 - DNA, 126 bp, synthetic construct
  source 1..126: mol_type = other DNA
ctgcagaacccctgctacatcggcacccacggctgcgacaccaacgccgcctgcagacccggccccagaacccagttcacctgcgagtgcagcatcggcttcagaggcgacggcagaacctgctac

SEQ ID NO: 48 - DNA, 126 bp, synthetic construct
  source 1..126: mol_type = other DNA
gacatcgacgagtgcagcgagcagcccagcgtgtgcggcagccacaccatctgcaacaaccaccccggcaccttcagatgcgagtgcgtggagggctaccagttcagcgacgagggcacctgcgtg

SEQ ID NO: 49 - DNA, 132 bp, synthetic construct
  source 1..132: mol_type = other DNA
cccatcaactactgcgagaccggcctgcacaactgcgacatcccccagagagcccagtgcatctacaccggcggcagcagctacacctgcagctgcctgcccggcttcagcggcgacggccaggcctgccag

SEQ ID NO: 50 - DNA, 117 bp, synthetic construct
  source 1..117: mol_type = other DNA
gacgtggacgagtgccagcccagcagatgccaccccgacgccttctgctacaacacccccggcagcttcacctgccagtgcaagcccggctaccagggcgacggcttcagatgcgtg

SEQ ID NO: 51 - DNA, 222 bp, synthetic construct
  source 1..222: mol_type = other DNA
aagaccagatgccagcacgagagagagcacatcctgggcgccgccggcgccaccgacccccagagacccatcccccccggcctgttcgtgcccgagtgcgacgcccacggccactacgcccccacccagtgccacggcagcaccggctactgctggtgcgtggacagagacggcagagaggtggagggcaccagaaccagacccggcatgacccccccctgc

SEQ ID NO: 52 - DNA, 863 bp, synthetic construct
  source 1..863: mol_type = other DNA
ctgagcaccgtggccccccccatccaccagggccccgccgtgcccaccgccgtgatccccctgccccccggcacccacctgctgttcgcccagaccggcaagatcgagagactgcccctggagggcaacaccatgagaaagaccgaggccaaggccttcctgcacgtgcccgccaaggtgatcatcggcctggccttcgactgcgtggacaagatggtgtactggaccgacatcaccgagcccagcatcggcagagccagcctgcacggcggcgagcccaccaccatcatcagacaggacctgggcagccccgagggcatcgccgtggaccacctgggcagaaacatcttctggaccgacagcaacctggacagaatcgaggtggccaagctggacggcacccagagaagagtgctgttcgagaccgacctggtgaaccccagaggcatcgtgaccgacagcgtgagaggcaacctgtactggaccgactggaacagagacaaccccaagatcgagaccagctacatggacggcaccaacagaagaatcctggtgcaggacgacctgggcctgcccaacggcctgaccttcgacgccttcagcagccagctgtgctgggtggacgccggcaccaacagagccgagtgcctgaaccccagccagcccagcagaagaaaggccctggagggcctgcagtaccccttcgccgtgaccagctacggcaagaacctgtacttcaccgactggaagatgaacagcgtggtggccctggacctggccatcagcaaggagaccgacgccttccagccccacaagcagaccagactgtacggcatcaccaccgccctgagccagtgccccca

SEQ ID NO: 53 - DNA, 111 bp, synthetic construct
  source 1..111: mol_type = other DNA
ggccacaactactgcagcgtgaacaacggcggctgcacccacctgtgcctggccacccccggcagcagaacctgcagatgccccgacaacaccctgggcgtggactgcatc

SEQ ID NO: 54 - DNA, 4122 bp, synthetic construct
  source 1..4122: mol_type = other DNA
atgagaggcggcgtgctgctggtgctgctgctgtgcgtggccgcccagtgcagacagagaggcctgttccccgccatcctgaacctggccagcaacgcccacatcagcaccaacgccacctgcggcgagaagggccccgagatgttctgcaagctggtggagcacgtgcccggcagacccgtgagaaacccccagtgcagaatctgcgacggcaacagcgccaaccccagagagagacaccccatcagccacgccatcgacggcaccaacaactggtggcagagccccagcatccagaacggcagagagtaccactgggtgaccatcaccctggacctgagacaggtgttccaggtggcctacgtgatcatcaaggccgccaacgcccccagacccggcaactggatcctggagagaagcctggacggcaccaccttcagcccctggcagtactacgccgtgagcgacagcgagtgcctgagcagatacaacatcacccccagaagaggcccccccacctacagagccgacgacgaggtgatctgcaccagctactacagcagactggtgcccctggagcacggcgagatccacaccagcctgatcaacggcagacccagcgccgacgacctgagccccaagctgctggagttcaccagcgccagatacatcagactgagactgcagagaatcagaaccctgaacgccgacctgatgaccctgagccacagagagcccaaggagctggaccccatcgtgaccagaagatactactacagcatcaaggacatcagcgtgggcggcatgtgcatctgctacggccacgccagcagctgcccctgggacgagaccaccaagaagctgcagtgccagtgcgagcacaacacctgcggcgagagctgcaacagatgctgccccggctaccaccagcagccctggagacccggcaccgtgagcagcggcaacacctgcgaggcctgcaactgccacaacaaggccaaggactgctactacgacgagagcgtggccaagcagaagaagagcctgaacaccgccggccagttcagaggcggcggcgtgtgcatcaactgcctgcagaacaccatgggcatcaactgcgagacctgcatcgacggctactacagaccccacaaggtgagcccctacgaggacgagccctgcagaccctgcaactgcgaccccgtgggcagcctgagcagcgtgtgcatcaaggacgacctgcacagcgacctgcacaacggcaagcagcccggccagtgcccctgcaaggagggctacaccggcgagaagtgcgacagatgccagctgggctacaaggactaccccacctgcgtgagctgcggctgcaaccccgtgggcagcgccagcgacgagccctgcaccggcccctgcgtgtgcaaggagaacgtggagggcaaggcctgcgacagatgcaagcccggcttctacaacctgaaggagaagaaccccagaggctgcagcgagtgcttctgcttcggcgtgagcgacgtgtgcagacagacctgcgccaacaacagacaccagtgcagcgtgcacgccgagtgcagagactacgccaccggcttctgctgcagctgcgtggccggctacaccggcaacggcagacagtgcgtggccgagggcagcccccagagagtgaacggcaaggtgaagggcagaatcttcgtgggcagcagccaggtgcccatcgtgttcgagaacaccgacctgcacagctacgtggtgatgaaccacggcagaagctacaccgccatcagcaccatccccgagaccgtgggctacagcctgctgcccctggcccccgtgggcggcatcatcggctggatgttcgccgtggagcaggacggcttcaagaacggcttcagcatcaccggcggcgagttcaccagacaggccgaggtgaccttcgtgggccaccccggcaacctggtgatcaagcagagattcagcggcatcgacgagcacggccacctgaccatcgacaccgagctggagggcagagtgccccagatccccttcggcagcagcgtgcacatcgagccctacaccgagctgtaccactacagcaccagcgtgatcaccagcagcagcaccagagagtacaccgtgaccgagcccgagagagacggcgccagccccagcagaatctacacctaccagtggagacagaccatcaccttccaggagtgcgtgcacgacgacagcagacccgccctgcccagcacccagcagctgagcgtggacagcgtgttcgtgctgtacaaccaggaggagaagatcctgagatacgccctgagcaacagcatcggccccgtgagagagggcagccccgacgccctgcagaacccctgctacatcggcacccacggctgcgacaccaacgccgcctgcagacccggccccagaacccagttcacctgcgagtgcagcatcggcttcagaggcgacggcagaacctgctacgacatcgacgagtgcagcgagcagcccagcgtgtgcggcagccacaccatctgcaacaaccaccccggcaccttcagatgcgagtgcgtggagggctaccagttcagcgacgagggcacctgcgtggccgtggtggaccagagacccatcaactactgcgagaccggcctgcacaactgcgacatcccccagagagcccagtgcatctacaccggcggcagcagctacacctgcagctgcctgcccggcttcagcggcgacggccaggcctgccaggacgtggacgagtgccagcccagcagatgccaccccgacgccttctgctacaacacccccggcagcttcacctgccagtgcaagcccggctaccagggcgacggcttcagatgcgtgcccggcgaggtggagaagaccagatgccagcacgagagagagcacatcctgggcgccgccggcgccaccgacccccagagacccatcccccccggcctgttcgtgcccgagtgcgacgcccacggccactacgcccccacccagtgccacggcagcaccggctactgctggtgcgtggacagagacggcagagaggtggagggcaccagaaccagacccggcatgacccccccctgcctgagcaccgtggccccccccatccaccagggccccgccgtgcccaccgccgtgatccccctgccccccggcacccacctgctgttcgcccagaccggcaagatcgagagactgcccctggagggcaacaccatgagaaagaccgaggccaaggccttcctgcacgtgcccgccaaggtgatcatcggcctggccttcgactgcgtggacaagatggtgtactggaccgacatcaccgagcccagcatcggcagagccagcctgcacggcggcgagcccaccaccatcatcagacaggacctgggcagccccgagggcatcgccgtggaccacctgggcagaaacatcttctggaccgacagcaacctggacagaatcgaggtggccaagctggacggcacccagagaagagtgctgttcgagaccgacctggtgaaccccagaggcatcgtgaccgacagcgtgagaggcaacctgtactggaccgactggaacagagacaaccccaagatcgagaccagctacatggacggcaccaacagaagaatcctggtgcaggacgacctgggcctgcccaacggcctgaccttcgacgccttcagcagccagctgtgctgggtggacgccggcaccaacagagccgagtgcctgaaccccagccagcccagcagaagaaaggccctggagggcctgcagtaccccttcgccgtgaccagctacggcaagaacctgtacttcaccgactggaagatgaacagcgtggtggccctggacctggccatcagcaaggagaccgacgccttccagccccacaagcagaccagactgtacggcatcaccaccgccctgagccagtgcccccagggccacaactactgcagcgtgaacaacggcggctgcacccacctgtgcctggccacccccggcagcagaacctgcagatgccccgacaacaccctgggcgtggactgcatcgagcagaagtga

SEQ ID NO: 55 - DNA, 383 bp, synthetic construct
  source 1..383: mol_type = other DNA
cgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattc

SEQ ID NO: 56 - DNA, 232 bp, synthetic construct
  source 1..232: mol_type = other DNA
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 57 - DNA, 798 bp, synthetic construct
  source 1..798: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 58 - DNA, 450 bp, synthetic construct
  source 1..450: mol_type = other DNA
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 59 - DNA, 436 bp, synthetic construct
  source 1..436: mol_type = other DNA
tgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 60 - DNA, 589 bp, synthetic construct
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 61 - DNA, 771 bp, synthetic construct
  source 1..771: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccag

SEQ ID NO: 62 - protein, 2045 aa, Homo sapiens
MAGRSHPGPLRPLLPLLVVAACVLPGAGGTCPERALERREEEANVVLTGTVEEILNVDPVQHTYSCKVRVWRYLKGKDLVARESLLDGGNKVVISGFGDPLICDNQVSTGDTRIFFVNPAPPYLWPAHKNELMLNSSLMRITLRNLEEVEFCVEDKPGTHFTPVPPTPPDACRGMLCGFGAVCEPNAEGPGRASCVCKKSPCPSVVAPVCGSDASTYSNECELQRAQCSQQRRIRLLSRGPCGSRDPCSNVTCSFGSTCARSADGLTASCLCPATCRGAPEGTVCGSDGADYPGECQLLRRACARQENVFKKFDGPCDPCQGALPDPSRSCRVNPRTRRPEMLLRPESCPARQAPVCGDDGVTYENDCVMGRSGAARGLLLQKVRSGQCQGRDQCPEPCRFNAVCLSRRGRPRCSCDRVTCDGAYRPVCAQDGRTYDSDCWRQQAECRQQRAIPSKHQGPCDQAPSPCLGVQCAFGATCAVKNGQAACECLQACSSLYDPVCGSDGVTYGSACELEATACTLGREIQVARKGPCDRCGQCRFGALCEAETGRCVCPSECVALAQPVCGSDGHTYPSECMLHVHACTHQISLHVASAGPCETCGDAVCAFGAVCSAGQCVCPRCEHPPPGPVCGSDGVTYGSACELREAACLQQTQIEEARAGPCEQAECGSGGSGSGEDGDCEQELCRQRGGIWDEDSEDGPCVCDFSCQSVPGSPVCGSDGVTYSTECELKKARCESQRGLYVAAQGACRGPTFAPLPPVAPLHCAQTPYGCCQDNITAARGVGLAGCPSACQCNPHGSYGGTCDPATGQCSCRPGVGGLRCDRCEPGFWNFRGIVTDGRSGCTPCSCDPQGAVRDDCEQMTGLCSCKPGVAGPKCGQCPDGRALGPAGCEADASAPATCAEMRCEFGARCVEESGSAHCVCPMLTCPEANATKVCGSDGVTYGNECQLKTIACRQGLQISIQSLGPCQEAVAPSTHPTSASVTVTTPGLLLSQALPAPPGALPLAPSSTAHSQTTPPPSSRPRTTASVPRTTVWPVLTVPPTAPSPAPSLVASAFGESGSTDGSSDEELSGDQEASGGGSGGLEPLEGSSVATPGPPVERASCYNSALGCCSDGKTPSLDAEGSNCPATKVFQGVLELEGVEGQELFYTPEMADPKSELFGETARSIESTLDDLFRNSDVKKDFRSVRLRDLGPGKSVRAIVDVHFDPTTAFRAPDVARALLRQIQVSRRRSLGVRRPLQEHVRFMDFDWFPAFITGATSGAIAAGATARATTASRLPSSAVTPRAPHPSHTSQPVAKTTAAPTTRRPPTTAPSRVPGRRPPAPQQPPKPCDSQPCFHGGTCQDWALGGGFTCSCPAGRGGAVCEKVLGAPVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGECGDHPCLPNPCHGGAPCQNLEAGRFHCQCPPGRVGPTCADEKSPCQPNPCHGAAPCRVLPEGGAQCECPLGREGTFCQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPVPHTVLNLKEPLYVGGAPDFSKLARAAAVSSGFDGAIQLVSLGGRQLLTPEHVLRQVDVTSFAGHPCTRASGHPCLNGASCVPREAAYVCLCPGGFSGPHCEKGLVEKSAGDVDTLAFDGRTFVEYLNAVTESEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 63 - protein, 3075 aa, Homo sapiens
MRGGVLLVLLLCVAAQCRQRGLFPAILNLASNAHISTNATCGEKGPEMFCKLVEHVPGRPVRNPQCRICDGNSANPRERHPISHAIDGTNNWWQSPSIQNGREYHWVTITLDLRQVFQVAYVIIKAANAPRPGNWILERSLDGTTFSPWQYYAVSDSECLSRYNITPRRGPPTYRADDEVICTSYYSRLVPLEHGEIHTSLINGRPSADDLSPKLLEFTSARYIRLRLQRIRTLNADLMTLSHREPKELDPIVTRRYYYSIKDISVGGMCICYGHASSCPWDETTKKLQCQCEHNTCGESCNRCCPGYHQQPWRPGTVSSGNTCEACNCHNKAKDCYYDESVAKQKKSLNTAGQFRGGGVCINCLQNTMGINCETCIDGYYRPHKVSPYEDEPCRPCNCDPVGSLSSVCIKDDLHSDLHNGKQPGQCPCKEGYTGEKCDRCQLGYKDYPTCVSCGCNPVGSASDEPCTGPCVCKENVEGKACDRCKPGFYNLKEKNPRGCSECFCFGVSDVCSSLSWPVGQVNSMSGWLVTDLISPRKIPSQQDALGGRHQVSINNTAVMQRLAPKYYWAAPEAYLGNKLTAFGGFLKYTVSYDIPVETVDSNLMSHADVIIKGNGLTLSTQAEGLSLQPYEEYLNVVRLVPENFQDFHSKRQIDRDQLMTVLANVTHLLIRANYNSAKMALYRLESVSLDIASSNAIDLVVAADVEHCECPQGYTGTSCESCLSGYYRVDGILFGGICQPCECHGHAAECNVHGVCIACAHNTTGVHCEQCLPGFYGEPSRGTPGDCQPCACPLTIASNNFSPTCHLNDGDEVVCDWCAPGYSGAWCERCADGYYGNPTVPGESCVPCDCSGNVDPSEAGHCDSVTGECLKCLGNTDGAHCERCADGFYGDAVTAKNCRACECHVKGSHSAVCHLETGLCDCKPNVTGQQCDQCLHGYYGLDSGHGCRPCNCSVAGSVSDGCTDEGQCHCVPGVAGKRCDRCAHGFYAYQDGSCTPCDCPHTQNTCDPETGECVCPPHTQGVKCEECEDGHWGYDAEVGCQACNCSLVGSTHHRCDVVTGHCQCKSKFGGRACDQCSLGYRDFPDCVPCDCDLRGTSGDACNLEQGLCGCVEETGACPCKENVFGPQCNECREGTFALRADNPLGCSPCFCSGLSHLCSELEDYVRTPVTLGSDQPLLRVVSQSNLRGTTEGVYYQAPDFLLDAATVRQHIRAEPFYWRLPQQFQGDQLMAYGGKLKYSVAFYSLDGVGTSNFEPQVLIKGGRIRKQVIYMDAPAPENGVRQEQEVAMRENFWKYFNSVSEKPVTREDFMSVLSDIEYILIKASYGQGLQQSRISDISMEVGRKAEKLHPEEEVASLLENCVCPPGTVGFSCQDCAPGYHRGKLPAGSDRGPRPLVAPCVPCSCNNHSDTCDPNTGKCLNCGDNTAGDHCDVCTSGYYGKVTGSASDCALCACPHSPPASFSPTCVLEGDHDFRCDACLLGYEGKHCERCSSSYYGNPQTPGGSCQKCDCNPHGSVHGDCDRTSGQCVCRLGASGLRCDECEPRHILMETDCVSCDDECVGVLLNDLDEIGDAVLSLNLTGIIPVPYGILSNLENTTKYLQESLLKENMQKDLGKIKLEGVAEETDNLQKKLTRMLASTQKVNRATERIFKESQDLAIAIERLQMSITEIMEKTTLNQTLDEDFLLPNSTLQNMQQNGTSLLEIMQIRDFTQLHQNATLELKAAEDLLSQIQENYQKPLEELEVLKEAASHVLSKHNNELKAAEALVREAEAKMQESNHLLLMVNANLREFSDKKLHVQEEQNLTSELIVQGRGLIDAAAAQTDAVQDALEHLEDHQDKLLLWSAKIRHHIDDLVMHMSQRNAVDLVYRAEDHAAEFQRLADVLYSGLENIRNVSLNATSAAYVHYNIQSLIEESEELARDAHRTVTETSLLSESLVSNGKAAVQRSSRFLKEGNNLSRKLPGIALELSELRNKTNRFQENAVEITRQTNESLLILRAIPKGIRDKGAKTKELATSASQSAVSTLRDVAGLSQELLNTSASLSRVNTTLRETHQLLQDSTMATLLAGRKVKDVEIQANLLFDRLKPLKMLEENLSRNLSEIKLLISQARKQAASIKVAVSADRDCIRAYQPQISSTNYNTLTLNVKTQEPDNLLFYLGSSTASDFLAVEMRRGRVAFLWDLGSGSTRLEFPDFPIDDNRWHSIHVARFGNIGSLSVKEMSSNQKSPTKTSKSPGTANVLDVNNSTLMFVGGLGGQIKKSPAVKVTHFKGCLGEAFLNGKSIGLWNYIEREGKCRGCFGSSQNEDPSFHFDGSGYSVVEKSLPATVTQIIMLFNTFSPNGLLLYLGSYGTKDFLSIELFRGRVKVMTDLGSGPITLLTDRRYNNGTWYKIAFQRNRKQGVLAVIDAYNTSNKETKQGETPGASSDLNRLDKDPIYVGGLPRSRVVRRGVTTKSFVGCIKNLEISRSTFDLLRNSYGVRKGCLLEPIRSVSFLKGGYIELPPKSLSPESEWLVTFATTNSSGIILAALGGDVEKRGDREEAHVPFFSVMLIGGNIEVHVNPGDGTGLRKALLHAPTGTCSDGQAHSISLVRNRRIITVQLDENNPVEMKLGTLVESRTINVSNLYVGGIPEGEGTSLLTMRRSFHGCIKNLIFNLELLDFNSAVGHEQVDLDTCWLSERPKLAPDAEDSKLLPEPRAFPEQCVVDAALEYVPGAHQFGLTQNSHFILPFNQSAVRKKLSVELSIRTFASSGLIYYMAHQNQADYAVLQLHGGRLHFMFDLGKGRTKVSHPALLSDGKWHTVKTDYVKRKGFITVDGRESPMVTVVGDGTMLDVEGLFYLGGLPSQYQARKIGNITHSIPACIGDVTVNSKQLDKDSPVSAFTVNRCYAVAQEGTYFDGSGYAALVKEGYKVQSDVNITLEFRTSSQNGVLLGISTAKVDAIGLELVDGKVLFHVNNGAGRITAAYEPKTATVLCDGKWHTLQANKSKHRITLIVDGNAVGAESPHTQSTSVDTNNPIYVGGYPAGVKQKCLRSQTSFRGCLRKLALIKSPQVQSFDFSRAFELHGVFLHSCPGTES

SEQ ID NO: 64 - protein, 1247 aa, Homo sapiens
MLASSSRIRAAWTRALLLPLLLAGPVGCLSRQELFPFGPGQGDLELEDGDDFVSPALELSGALRFYDRSDIDAVYVTTNGIIATSEPPAKESHPGLFPPTFGAVAPFLADLDTTDGLGKVYYREDLSPSITQRAAECVHRGFPEISFQPSSAVVVTWESVAPYQGPSRDPDQKGKRNTFQAVLASSDSSSYAIFLYPEDGLQFHTTFSKKENNQVPAVVAFSQGSVGFLWKSNGAYNIFANDRESVENLAKSSNSGQQGVWVFEIGSPATTNGVVPADVILGTEDGAEYDDEDEDYDLATTRLGLEDVGTTPFSYKALRRGGADTYSVPSVLSPRRAATERPLGPPTERTRSFQLAVETFHQQHPQVIDVDEVEETGVVFSYNTDSRQTCANNRHQCSVHAECRDYATGFCCSCVAGYTGNGRQCVAEGSPQRVNGKVKGRIFVGSSQVPIVFENTDLHSYVVMNHGRSYTAISTIPETVGYSLLPLAPVGGIIGWMFAVEQDGFKNGFSITGGEFTRQAEVTFVGHPGNLVIKQRFSGIDEHGHLTIDTELEGRVPQIPFGSSVHIEPYTELYHYSTSVITSSSTREYTVTEPERDGASPSRIYTYQWRQTITFQECVHDDSRPALPSTQQLSVDSVFVLYNQEEKILRYALSNSIGPVREGSPDALQNPCYIGTHGCDTNAACRPGPRTQFTCECSIGFRGDGRTCYDIDECSEQPSVCGSHTICNNHPGTFRCECVEGYQFSDEGTCVAVVDQRPINYCETGLHNCDIPQRAQCIYTGGSSYTCSCLPGFSGDGQACQDVDECQPSRCHPDAFCYNTPGSFTCQCKPGYQGDGFRCVPGEVEKTRCQHEREHILGAAGATDPQRPIPPGLFVPECDAHGHYAPTQCHGSTGYCWCVDRDGREVEGTRTRPGMTPPCLSTVAPPIHQGPAVPTAVIPLPPGTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLTFDAFSSQLCWVDAGTNRAECLNPSQPSRRKALEGLQYPFAVTSYGKNLYFTDWKMNSVVALDLAISKETDAFQPHKQTRLYGITTALSQCPQGHNYCSVNNGGCTHLCLATPGSRTCRCPDNTLGVDCIEQK

SEQ ID NO: 65 - protein, 10 aa, synthetic construct
KGLVEASAGD

SEQ ID NO: 66 - DNA, 21 bp, synthetic construct
  source 1..21: mol_type = other DNA
ggaacccctagtgatggagtt

SEQ ID NO: 67 - DNA, 16 bp, synthetic construct
  source 1..16: mol_type = other DNA
cggcctcagtgagcga

SEQ ID NO: 68 - protein, 1002 aa, synthetic construct
MRGGVLLVLLLCVAAQCRQRGLFPAILNLASNAHISTNATCGEKGPEMFCKLVEHVPGRPVRNPQCRICDGNSANPRERHPISHAIDGTNNWWQSPSIQNGREYHWVTITLDLRQVFQVAYVIIKAANAPRPGNWILERSLDGTTFSPWQYYAVSDSECLSRYNITPRRGPPTYRADDEVICTSYYSRLVPLEHGEIHTSLINGRPSADDLSPKLLEFTSARYIRLRLQRIRTLNADLMTLSHREPKELDPIVTRRYYYSIKDISVGGMCICYGHASSCPWDETTKKLQCQCEHNTCGESCNRCCPGYHQQPWRPGTVSSGNTCEACNCHNKAKDCYYDESVAKQKKSLNTAGQFRGGGVCINCLQNTMGINCETCIDGYYRPHKVSPYEDEPCRPCNCDPVGSLSSVCIKDDLHSDLHNGKQPGQCPCKEGYTGEKCDRCQLGYKDYPTCVSCGCNPVGSASDEPCTGPCVCKENVEGKACDRCKPGFYNLKEKNPRGCSECFCFGVSDVCPINYCETGLHNCDIPQRAQCIYTGGSSYTCSCLPGFSGDGQACQDVDECQPSRCHPDAFCYNTPGSFTCQCKPGYQGDGFRCVPGEVEKTRCQHEREHILGAAGATDPQRPIPPGLFVPECDAHGHYAPTQCHGSTGYCWCVDRDGREVEGTRTRPGMTPPCLSTVAPPIHQGPAVPTAVIPLPPGTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLTFDAFSSQLCWVDAGTNRAECLNPSQPSRRKALEGLQYPFAVTSYGKNLYFTDWKMNSVVALDLAISKETDAFQPHKQTRLYGITTALSQCPQGHNYCSVNNGGCTHLCLATPGSRTCRCPDNTLGVDCIEQK

SEQ ID NO: 69 - DNA, 3009 bp, synthetic construct
  source 1..3009: mol_type = other DNA
atgagaggcggcgtgctgctggtgctgctgctgtgcgtggccgcccagtgcagacagagaggcctgttccccgccatcctgaacctggccagcaacgcccacatcagcaccaacgccacctgcggcgagaagggccccgagatgttctgcaagctggtggagcacgtgcccggcagacccgtgagaaacccccagtgcagaatctgcgacggcaacagcgccaaccccagagagagacaccccatcagccacgccatcgacggcaccaacaactggtggcagagccccagcatccagaacggcagagagtaccactgggtgaccatcaccctggacctgagacaggtgttccaggtggcctacgtgatcatcaaggccgccaacgcccccagacccggcaactggatcctggagagaagcctggacggcaccaccttcagcccctggcagtactacgccgtgagcgacagcgagtgcctgagcagatacaacatcacccccagaagaggcccccccacctacagagccgacgacgaggtgatctgcaccagctactacagcagactggtgcccctggagcacggcgagatccacaccagcctgatcaacggcagacccagcgccgacgacctgagccccaagctgctggagttcaccagcgccagatacatcagactgagactgcagagaatcagaaccctgaacgccgacctgatgaccctgagccacagagagcccaaggagctggaccccatcgtgaccagaagatactactacagcatcaaggacatcagcgtgggcggcatgtgcatctgctacggccacgccagcagctgcccctgggacgagaccaccaagaagctgcagtgccagtgcgagcacaacacctgcggcgagagctgcaacagatgctgccccggctaccaccagcagccctggagacccggcaccgtgagcagcggcaacacctgcgaggcctgcaactgccacaacaaggccaaggactgctactacgacgagagcgtggccaagcagaagaagagcctgaacaccgccggccagttcagaggcggcggcgtgtgcatcaactgcctgcagaacaccatgggcatcaactgcgagacctgcatcgacggctactacagaccccacaaggtgagcccctacgaggacgagccctgcagaccctgcaactgcgaccccgtgggcagcctgagcagcgtgtgcatcaaggacgacctgcacagcgacctgcacaacggcaagcagcccggccagtgcccctgcaaggagggctacaccggcgagaagtgcgacagatgccagctgggctacaaggactaccccacctgcgtgagctgcggctgcaaccccgtgggcagcgccagcgacgagccctgcaccggcccctgcgtgtgcaaggagaacgtggagggcaaggcctgcgacagatgcaagcccggcttctacaacctgaaggagaagaaccccagaggctgcagcgagtgcttctgcttcggcgtgagcgacgtgtgccccatcaactactgcgagaccggcctgcacaactgcgacatcccccagagagcccagtgcatctacaccggcggcagcagctacacctgcagctgcctgcccggcttcagcggcgacggccaggcctgccaggacgtggacgagtgccagcccagcagatgccaccccgacgccttctgctacaacacccccggcagcttcacctgccagtgcaagcccggctaccagggcgacggcttcagatgcgtgcccggcgaggtggagaagaccagatgccagcacgagagagagcacatcctgggcgccgccggcgccaccgacccccagagacccatcccccccggcctgttcgtgcccgagtgcgacgcccacggccactacgcccccacccagtgccacggcagcaccggctactgctggtgcgtggacagagacggcagagaggtggagggcaccagaaccagacccggcatgacccccccctgcctgagcaccgtggccccccccatccaccagggccccgccgtgcccaccgccgtgatccccctgccccccggcacccacctgctgttcgcccagaccggcaagatcgagagactgcccctggagggcaacaccatgagaaagaccgaggccaaggccttcctgcacgtgcccgccaaggtgatcatcggcctggccttcgactgcgtggacaagatggtgtactggaccgacatcaccgagcccagcatcggcagagccagcctgcacggcggcgagcccaccaccatcatcagacaggacctgggcagccccgagggcatcgccgtggaccacctgggcagaaacatcttctggaccgacagcaacctggacagaatcgaggtggccaagctggacggcacccagagaagagtgctgttcgagaccgacctggtgaaccccagaggcatcgtgaccgacagcgtgagaggcaacctgtactggaccgactggaacagagacaaccccaagatcgagaccagctacatggacggcaccaacagaagaatcctggtgcaggacgacctgggcctgcccaacggcctgaccttcgacgccttcagcagccagctgtgctgggtggacgccggcaccaacagagccgagtgcctgaaccccagccagcccagcagaagaaaggccctggagggcctgcagtaccccttcgccgtgaccagctacggcaagaacctgtacttcaccgactggaagatgaacagcgtggtggccctggacctggccatcagcaaggagaccgacgccttccagccccacaagcagaccagactgtacggcatcaccaccgccctgagccagtgcccccagggccacaactactgcagcgtgaacaacggcggctgcacccacctgtgcctggccacccccggcagcagaacctgcagatgccccgacaacaccctgggcgtggactgcatcgagcagaagtga

SEQ ID NO: 70 - protein, 10 aa, synthetic construct
LGESPVPHTV

SEQ ID NO: 71 - protein, 10 aa, synthetic construct
AVTESEKALQ

SEQ ID NO: 72 - DNA, 1049 bp, synthetic construct
  source 1..1049: mol_type = other DNA
cctcttcagtcgatcatctgagaccttcacacttcctgttcagacttctttcctagtgaaccctttcaagagcccccacaccgaatcaaacatggatggcacaaacatggatgctctaaatctacacagagcttcacaaatgtcacacgtgcacacactttcacctcagctcttacctttgctgttccatctagccattgcccctcccgttccctttttcaaaatggctgctccttcaaggtctctagacaacactgcttcctagacctgattcctctttcctctgaacttcctgtattaagtggcattcccaccgttctgtgcattggcagttgatgagtttgccctctgtttctcccctctgcctcctccaactagatcttgagcttgtagaaagaactgaattaccattctaatgtgagcattcgcattccccaacgtttcaaatagccaccaggcaggccaatacgcagtcttgatcatttaaactgctgcattgcataaggtaccctaacagacgggagaaatctgggcttcttcctggaagtttcctggtagagaaaatcttctatctgggtagaatctaccagatacagggaggtggaaaaagttgttcccagagggctttatagtctacagtgttgccgtagcaatcagaacaacagacaccctaatttgggagtgctctgaaagaaacttgcctctaggtcctagggctctcaggcaaggagaccaagaaggattcctttgccatggccttttggatttaggtttctcagcttgtctattcctcagagaagtgtgtctatgccccttttctgtccctctgcctcaccctaccccaacattccaacctagggtagggggaggtcagtatacacaaagccctctgtgtaaggggtggtatgtgtccccccacccctccacccagcgtatacaatgccccttctgctccatgccccctgccaccctccccaccacctctccattgcacatgccaggctgcaattggtcactggctcaggacagccccctcatgctggggatcc